(19) United States
(12) Patent Application Publication
(10) Pub. No.: US 2012/0021427 A1
(43) Pub. Date: Jan. 26, 2012

(54) Methods For Rapid Forensic DNA Analysis

(75) Inventors: Steven A. Hofstadler, Vista, CA (US); Thomas A. Hall, Oceanside, CA (US)

(73) Assignee: IBIS BIOSCIENCE, INC, Carlsbad, CA (US)

(21) Appl. No.: 13/259,406

(22) PCT Filed: May 6, 2010
(86) PCT No.: PCT/US10/33898

§ 371 Date: Sep. 23, 2011

Related U.S. Application Data
(60) Provisional application No. 61/176,028, filed on May 6, 2009.

(51) Int. Cl. C12Q 1/68; C07H 21/04

(52) U.S. Cl. 435/6.12; 435/6.1; 536/24.33

(57) ABSTRACT

The present invention provides methods and primer pairs for rapid, high-resolution forensic analysis of DNA and STR-typing by using amplification and mass spectrometry, determining the molecular masses and calculating base compositions of amplification products and comparing the molecular masses with the molecular masses of theoretical amplicons indexed in a database.

### SEQUENCE LISTING

The present application is being filed along with a Sequence Listing in electronic format. The Sequence Listing is provided as a file entitled 9936WOO1.txt. The information in the electronic format of the sequence listing is incorporated herein by reference in its entirety.

### FIELD OF THE INVENTION

This invention relates generally to the fields of genetic mapping and genetic identity testing, including forensic testing and paternity testing. In certain aspects, the invention relates to the use of amplification and mass spectrometry in DNA analysis using tandem repeat regions of DNA. In other aspects, the invention provides for rapid and accurate forensic analysis by using mass spectrometry to characterize informative regions of DNA.

### BACKGROUND OF THE INVENTION

The process of human identification through DNA analysis is a common objective of forensics investigations. As used herein, “forensics” is the study of evidence, for example, that discovered at a crime or accident scene that is then used in a court of law. “Forensic science” is any science used to answer questions of interest to the legal system, in particular the criminal or civil justice system, providing impartial scientific evidence for use in the courts of law, for example, in criminal investigations and trials. Forensic science is a multidisciplinary subject, drawing principally from chemistry and biology, but also from physics, geology, psychology and social science, for example. The goal of one aspect of human forensics, forensic DNA typing, is to determine the identity or genotype of DNA acquired from a forensic sample, for example, evidence from a crime scene or DNA sample from an individual. Typical sources of such DNA evidence include hair, bones, teeth, and body fluids such as saliva, semen, and blood. There often exists a need for rapid identification of a large number of humans, human remains and/or biological samples. Such remains or samples may be associated with war-related casualties, aircraft crashes, and acts of terrorism, for example.

Tandem DNA repeat regions, which are prevalent in the human genome and exhibit a high degree of variability among individuals, are used in a number of fields, including human forensics and identity testing, genetic mapping, and linkage analysis. Various types of DNA repeat regions exist within eukaryotic genomes and can be classified based on length of their core repeat regions. Short tandem repeats (STRs), also called simple sequence repeats (SSRs), or microsatellites are repeat regions having core units of between 2-6 nucleotides in length. For a particular STR locus, individuals in a population differ in the number of these core repeat units.

STR typing involves the amplification of multiple STR DNA loci that display a collection of alleles in the human population that differ in repeat number. Typically, the products of such amplification reactions are analyzed by polyacrylamide gel or capillary electrophoresis using fluorescent detection methods, and subsequent discrimination among different alleles based on amplification product length. Because a typical STR typing analysis will use multiple STR loci that are not genetically linked, the product rule can be applied to estimate the probability of a random match to any STR profile where population allele frequencies have been characterized for each locus (Holt C L, et. al. (2000) Forensic Sci. Int. 112(2-3): 91-109; Holland M M, et. al. (2003) Croat. Med. J. 44(3): 264-72). This leads to extremely high differentiation power with low random match probabilities within the human population. Because of the short length of STR repeats and the high degree of variability in number of repeats among individuals in a population, STR typing has become a standard in human forensics where sufficient nuclear DNA is available.

A number of tetranucleotide STRs and methods for STR-typing have been explored for application in human forensics. Commercial STR-typing kits are available that target different STR loci, including a common set of loci. The FBI Laboratory has established 13 nationally recognized core STR loci that are included in a national forensic DNA database known as the Combined DNA Index System (CODIS). The 13 CODIS core loci are CSF1PO, FGA, TH01, TPOX, VWA, D3S1358, D5S818, D7S820, D8S1179, D13S317, D16S539, D18S51, and D21S11. Sequence information for these loci are available from STRBase. The range of numbers of repeat units for reported alleles for these CODIS 13 loci is 6-16, 15-51.2, 3-14, 6-13, 10-24, 9-20, 7-16, 6-15, 8-19, 5-15, 5-15, 7-27, and 24-38 respectively (Butler, J M, 2001 Forensic DNA Typing Academic Press). When profiles are available with allele information for all 13 of these core STR loci, the average probability of a random match is lower than one in a trillion among non-related individuals. STR-typing by DNA sequencing is less desirable as it presents time constraints and is labor intensive.

Y-STRs are STRs located on the Y chromosome and are designated by “DYS numbers” where “DYS” refers to “DNA Y chromosome Segment.” A core group of minimum haplotype markers has been defined which includes DYS393, DYS19, DYS391, DYS385a/b, DYS390, DYS392, DYS437, DYS438, DYS439, and DYS389I/II (Butler, J. M. Forensic DNA Typing, 2nd ed.; Elsevier Academic Press: Burlington, 2005). Y-STRs have been used by forensic laboratories to examine sexual assault evidence. In a sexual assault case, evidence will contain both female and male DNA. Differential extraction is often used to separate the male component from the female component. More often, however, the male and female components cannot be separated completely. As a result, the female component could exist prominently even in the male component after separation. When the “male DNA sample” undergoes the PCR amplification process, the female DNA component is amplified as well, sometimes masking the male DNA, which makes analysis difficult. Masking does not occur when Y-STRs are examined Since there is no Y-STR in the female evidence, Y-STR data can only come from the assailant(s) in such a sexual assault case. The male component will be easily detected, since only this part of DNA will be amplified. The Y-STR system is especially helpful in cases with more man one assailant. The mixed pattern in the evidence can help to identify those males responsible for the assault. Y-STR analysis is also used for non-sexual assault cases where mixed samples are collected from evidence. A conventional STR analysis will often cause the masking effect if there is a very small quantity of male DNA in the mixed sample. Performing Y-STR testing can help to identify all males who have contributed to the evidence.

STR-typing using STR markers has become the human forensic “gold standard” as the combined information derived from the 13 distinct CODIS alleles provide enough information to uniquely identify an individual's DNA signature to a statistical significance of 1 in 10⁹. Standard or conventional STR-typing methods, which typically use amplification and electrophoretic size determination to resolve individual alleles, have certain limitations. At low STR copy number it is not uncommon to observe allele “drop out” in which a heterozygous individual is typed as a homozygote because one of the alleles is not detected. Additionally, in cases of highly degraded or low copy DNA samples, entire markers may drop out leaving only a few STRs from which to derive a DNA profile. In certain situations for example, such as mass disaster victim identification, a large number of samples with varying DNA quantity and quality can exist, many of which produce only partial STR profiles. While in some cases a partial profile can be used to include or exclude a potential suspect or identity, conventional STR typing methods sometimes do not provide sufficient resolution at the available loci in the case of a partial profile. Thus, there is a need within the forensics community to increase resolution of STR-typing methods, such that it is possible to derive additional information from degraded DNA samples which yield an incomplete set of STR markers and from other samples where detection of the complete STR set is not possible.

Techniques would be beneficial that could resolve sequence polymorphisms in alleles and thus increase the observed allelic variation for several common STR loci, while maintaining the advantages of amplification-based techniques, such as rapidness and the ability to automate the procedure for high-throughput typing. Thus, there is a need for STR typing methods that provide a higher level of resolution compared with standard techniques. Moreover, there exists a need for the development of an automated platform capable of high-throughput sample processing to enable analysis of a large number of samples produced simultaneously or over a short period of time, as in the case of mass disaster or war.

Mass spectrometry provides detailed information about the molecules being analyzed, including high mass accuracy. It is also a process that can be easily automated. Electrospray ionization mass spectrometry (ESI-MS) provides a platform capable of automated sample processing, and can resolve sequence polymorphisms between STR alleles (Ecker et. al. J. Assoc. Laboratory Automation 2006, 11, 341-51).

Matrix-assisted laser desorption-ionization time-of-flight mass spectrometry (MALDI TOF MS) has been employed to analyze STR, SNP, and Y-chromosome markers. (Butler, J.; Becker, C. H. Science and Technology Research Report to NIJ 2001, NCJ 188292, October; Monforte, J. A.; Becker, C. H. Nat Med 1997, 3, 360-362; Taranenko, N. I.; Golovlev, V. V.; Allman, S. L.; Taranenko, N. V.; Chen, C. H.; Hong, J.; Chang, L. Y. Rapid Commun Mass Spectrom 1998, 12, 413-418; Butler, J. M.; Li, J.; Shaler, T. A.; Monforte, J. A.; Becker, C. H. Int J Legal Med 1999, 112, 45-49; Ross, P. L.; Belgrader, P. Anal Chem 1997, 69, 3966-3972). To obtain routinely the necessary mass accuracy and resolution using MALDI TOF MS, the amplicon size must be less than 100 bp, which often requires strategies such as enzymatic digestion and nested linear amplification. In the MALDI approach, PCR amplicons must be thoroughly desalted and co-crystallized with a suitable matrix prior to mass spectrometric analysis. The size reduction schemes and clean-up schemes employed for STR and SNP analyses in the cited reports resulted in the mass spectrometric analysis of only one strand of the PCR amplicon. By measuring the mass of only one strand of the amplicon, an unambiguous base composition may be difficult to determine and only the length of the allele may be obtained. Even with the size reduction schemes, mass measurement errors of 12 to 60 Daltons (Da) are observed for products in the size range 15000 to 25000 Da. This corresponds to mass measurement errors of the 800 to 2400 ppm. Because of poor mass accuracy and mass resolution typical of MALDI, multiplexing of STRs is difficult and not routine, although in one published report three STR loci were successfully multiplexed. The issue of allelic balance has not been addressed for MALDI-TOF-MS based assays.

U.S. Pat. Nos. 6,764,822 and 6,090,558 relate to methods for STR-typing using mass spectrometry (MS). Use of electrospray ionization (ESI)-MS to resolve STR alleles has been reported (Hannis and Muddiman, 2001, Rapid Commun. Mass. Spectrom. 15(5): 348-50; Hannis et. al, 2000, Advances in Nucleic Acid and Protein Analysis, Manipulation and Sequencing, 3926: 1017-2661). ESI-MS provides a platform capable of automated sample processing and analysis that can resolve sequence polymorphisms (Ecker et. al. (2006) JALA. 11:341-51).

Several groups have described detection of PCR products using high resolution electrospray ionization-Fourier transform-ion cyclotron resonance mass spectrometry (ESI-FT-ICR MS). Accurate measurement of exact mass combined with knowledge of the number of at least one nucleotide allowed calculation of the total base composition for PCR duplex products of approximately 100 base pairs. (Aaserud et al., J. Am. Soc. Mass Spec., 1996, 7, 1266-1269; Muddiman et al., Anal. Chem., 1997, 69, 1543-1549; Wunschel et al., Anal. Chem., 1998, 70, 1203-1207; Muddiman et al., Rev. Anal. Chem., 1998, 17, 1-68). Electrospray ionization-Fourier transform-ion cyclotron resistance (ESI-FT-ICR) MS may be used to determine the mass of double-stranded, 500 base-pair PCR products via the average molecular mass (Hurst et al., Rapid Commun. Mass Spec. 1996, 10, 377-382).

There is an unmet need for methods and compositions for analysis of DNA forensic markers that approach the level of resolution sequencing affords, that is capable of scanning a substantial amount of the variation contained within an amplified fragment, yet that is also rapid, amenable to automation, and provides relevant information without the burden of extensive manual data interpretation. Preferably, such a method would not require a priori knowledge of the potentially informative sites within a sample to carry out an analysis. Preferably, such methods would be able to provide substantial resolving capability for forensic analyses in cases of degraded DNA or with relatively low amounts of DNA, for example, by allowing resolution of sequence polymorphisms that may allow discrimination of equal or same-length alleles based on small differences in sequence or base composition.

### SUMMARY OF THE INVENTION

The methods compositions and kits provided herein are directed to forensic analysis and identity testing based on using mass spectrometry to “weigh” DNA forensic markers with enough accuracy to yield an unambiguous base composition (i.e. the number of A's, G's, C's and T's) which in turn can be used to derive a DNA profile for an individual. Importantly, these base composition profiles can be referenced to existing forensics databases derived from STR or other forensic marker profiles. The present disclosure provides methods, primer pair compositions and kits that are capable of resolving human forensic DNA samples using STR loci based upon length and sequence polymorphisms, as measured by base composition, in a high throughput manner.

The present invention is directed to methods of forensic analysis of DNA. In some embodiments the methods comprise identity testing. In some embodiments they comprise STR-typing. The methods provided herein can be distinguished from conventional amplification based STR-typing. For example, the methods provided herein provide the ability to assign allele designations for STR loci based upon size as determined by mass. In addition, the methods provided herein can further resolve apparently similar alleles which differ only by one or more SNPs by deriving information from the loci nucleotide sequence as measured by mass or base composition uncovering additional alleles within the loci.

In some embodiments methods are provided for identifying a known STR allele or characterizing a previously unknown STR allele in a nucleic acid sample. A nucleic acid locus which includes the STR allele is selected and at least a portion of the locus is amplified using an oligonucleotide primer pair comprising a forward and a reverse primer, each between 13 and 40 nucleobases in length. An amplification product with a length of about 45 to about 200 nucleobases is thus generated. The amplification product duplicates the sequence of the known or unknown STR allele. The molecular mass of one or both strands of the amplification product is measured and the base composition of one or both of the strands is determined The base composition is then compared to a plurality of database-stored base compositions of strands of amplification products of known alleles of the locus. When a match is identified between the base composition and at least one of the database-stored base compositions of amplification products comprising the sequence of the STR allele produced with the primer pair, the allele is identified. Alternatively, when the comparison fails to identify a match between the base composition and at least one of the database-stored base compositions, a previously unknown STR allele is characterized. In a preferred embodiment, the locus is located on a human Y chromosome.

In some embodiments, the base composition of the previously unknown STR allele is added to the plurality of database stored base compositions. The base composition of the previously unknown STR allele may include a single nucleotide polymorphism relative to a known STR allele. The database-stored base compositions may include molecular masses which are calculated from theoretical amplification products of known sequences of known alleles and may also include measured molecular masses or actual amplification products of known sequences of known alleles or newly characterized alleles. Newly characterized alleles are, for example, alleles which have a SNP relative to a known allele.

In some embodiments, the step of measuring the molecular mass is performed by mass spectrometry, preferably ESI-TOF mass spectrometry.

In some embodiments, the forward primer and the reverse primer each comprise a thymidine reside at the 5′ end, thereby minimizing non-templated adenylation of the amplification product.

In another embodiment, the amplification is performed using deoxynucleotide triphosphates comprising ¹³C-enriched dGTP or a ¹³C-enriched analogue of dGTP. Preferably, this step is also performed using deoxynucleotide triphosphates comprising non-isotope enriched dCTP, dTTP and dATP.

In some embodiments, the locus is selected from the group consisting of DYS393, DYS19, DYS391, DYS385a/b, DYS390, DYS392, DYS437, DYS438, DYS439, DYS389I, and DYS389II.

In certain embodiments, the locus is DYS393. In one aspect, it is preferred if each member of the primer pair has at least 70%, at least 80%, at least 90%, at least 95% or at least 100% sequence identity with a corresponding member of a primer pair selected from the group consisting of: SEQ ID NOs: 1:43, 63:54, 67:12, 62:64, 62:55, 33:31 and 34:30, wherein, with respect to pairs of sequence identifiers (X:Y) for primer pairs, the convention as defined herein is that the sequence identifier to the left of the colon (X:) represents the forward primer and the sequence identifier to the right of the colon (:Y) represents the reverse primer. In another aspect, each member of the primer pair has at least 70%, at least 80%, at least 90%, at least 95% or at least 100% sequence identity with a corresponding member of: SEQ ID NOs: 63:54. In a preferred aspect, the primer pair is the primer pair of SEQ ID NOs: 63:54.

In some embodiments, the locus is DYS19. In one aspect, it is preferred if each member of the primer pair has at least 70%, at least 80%, at least 90%, at least 95% or at least 100% sequence identity with a corresponding member of a primer pair selected from the group consisting of: SEQ ID NOs: 16:28, 51:17 and 45:60. In another aspect, each member of the primer pair has at least 70%, at least 80%, at least 90%, at least 95% or at least 100% sequence identity with a corresponding member of: SEQ ID NOs: 51:17. In a preferred aspect, the primer pair is the primer pair of SEQ ID NOs: 51:17.

In some embodiments, the locus is DYS391. In one aspect, it is preferred if each member of the primer pair has at least 70%, at least 80%, at least 90%, at least 95% or at least 100% sequence 30 identity with a corresponding member of a primer pair selected from the group consisting of: SEQ ID NOs: 32:50, 19:13, 19:48, and 70:57. In another aspect, each member of the primer pair has at least 70%, at least 80%, at least 90%, at least 95% or at least 100% sequence identity with a corresponding member of: SEQ ID NOs: 19:48. In a preferred aspect, the primer pair is the primer pair of SEQ ID NOs: 19:48.

In certain embodiments, the locus is DYS385a/b. In one aspect, it is preferred if each member of the primer pair has at least 70%, at least 80%, at least 90%, at least 95% or at least 100% sequence identity with a corresponding member of a primer pair selected from the group consisting of: SEQ ID NOs: 10:27, 42:27, 10:35, 42:66 and 72:67. In another aspect, each member of the primer pair has at least 70%, at least 80%, at least 90%, at least 95% or at least 100% sequence identity with a corresponding member of: SEQ ID NOs: 72:67. In a preferred aspect, the primer pair is the primer pair of SEQ ID NOs: 72:67.

In some embodiments, the locus is DYS390. In one aspect, it is preferred if each member of the primer pair has at least 70%, at least 80%, at least 90%, at least 95% or at least 100% sequence identity with a corresponding member of a primer pair selected from the group consisting of: SEQ ID NOs: 59:20, 21:49, 59:49, 39:68 and 73:74. In another aspect, each member of the primer pair has at least 70%, at least 80%, at least 90%, at least 95% or at least 100% sequence identity with a corresponding member of: SEQ ID NOs: 39:68. In a preferred aspect, the primer pair is the primer pair of SEQ ID NOs: 39:68.

In certain embodiments, the locus is DYS392. In one aspect, it is preferred if each member of the primer pair has at least 70%, at least 80%, at least 90%, at least 95% or at least 100% sequence identity with a corresponding member of a primer pair selected from the group consisting of: SEQ ID NOs: 26:11, 53:29, 25:18, and 69:18. In another aspect, each member of the primer pair has at least 70%, at least 80%, at least 90%, at least 95% or at least 100% sequence identity with a corresponding member of: SEQ ID NOs: 53:29. In a preferred aspect, the primer pair is the primer pair of SEQ ID NOs: 53:29.

In some embodiments, the locus is DYS437. In one aspect, it is preferred if each member of the primer pair has at least 70%, at least 80%, at least 90%, at least 95% or at least 100% sequence identity with a corresponding member of a primer pair selected from the group consisting of: SEQ ID NOs: 65:44, 36:14, 8:14, 38:61, and 36:37. In another aspect, each member of the primer pair has at least 70%, at least 80%, at least 90%, at least 95% or at least 100% sequence identity with a corresponding member of: SEQ ID NOs: 36:37. In a preferred aspect, the primer pair is the primer pair of SEQ ID NOs: 36:37.

In some embodiments, the locus is DYS438. In one aspect, it is preferred if each member of the primer pair has at least 70%, at least 80%, at least 90%, at least 95% or at least 100% sequence identity with a corresponding member of a primer pair selected from the group consisting of: SEQ ID NOs: 7:56, 71:41, 22:6, and 71:9. In another aspect, each member of the primer pair has at least 70%, at least 80%, at least 90%, at least 95% or at least 100% sequence identity with a corresponding member of: SEQ ID NOs: 22:6. In a preferred aspect, the primer pair is the primer pair of SEQ ID NOs: 22:6.

In some embodiments, the locus is DYS439. In one aspect, it is preferred if each member of the primer pair has at least 70%, at least 80%, at least 90%, at least 95% or at least 100% sequence identity with a corresponding member of a primer pair selected from the group consisting of: SEQ ID NOs: 3:58, 2:40, 4:52, and 2:52. In another aspect, each member of the primer pair has at least 70%, at least 80%, at least 90%, at least 95% or at least 100% sequence identity with a corresponding member of: SEQ ID NOs: 4:52. In a preferred aspect, the primer pair is the primer pair of SEQ ID NOs: 4:52.

In certain embodiments, the locus is DYS389I. In one aspect, it is preferred if each member of the primer pair has at least 70%, at least 80%, at least 90%, at least 95% or at least 100% sequence identity with a corresponding member of a primer pair selected from the group consisting of: SEQ ID NOs: 23:15, and 23:5. In another aspect, each member of the primer pair has at least 70%, at least 80%, at least 90%, at least 95% or at least 100% sequence identity with a corresponding member of: SEQ ID NOs: 23:5. In a preferred aspect, the primer pair is the primer pair of SEQ ID NOs: 23:5.

In some embodiments, the locus is DYS389II. In one aspect, it is preferred if each member of the primer pair has at least 70%, at least 80%, at least 90%, at least 95% or at least 100% sequence identity with SEQ ID NO: 24:47. In a preferred aspect, the primer pair is the primer pair of SEQ ID NOs 24:47.

Another aspect is a purified oligonucleotide primer pair for identifying a known STR allele or characterizing a previously unknown STR allele in a nucleic acid sample. The primer pair is configured to produce an amplification product of at least a portion of an STR locus. The amplification product duplicates the sequence of the known STR allele or the previously unknown STR allele. Each member of the primer pair has at least 70%, at least 80%, at least 90%, at least 95% or at least 100% sequence identity with a corresponding member of a primer pair selected from the group consisting of: SEQ ID NOs: 16:28, 51:17, 45:60, 10:27, 42:27, 10:35, 24:46, 23:15, 23:5, 24:47, 59:20, 21:49, 59:49, 39:68, 32:50, 19:13, 19:48, 70:57, 26:11, 53:29, 25:18, 69:18, 1:43, 63:54, 67:12, 62:64, 65:44, 36:14, 8:14, 38:61, 36:37, 7:56, 71:41, 22:6, 71:9, 3:58, 2:40, 4:52, 2:52, 62:55, 33:31, 34:30, 73:74, 42:66 and 72:67.

At least one member of the primer pair may include a mass-modified nucleobase, a universal nucleobase, or a non-templated 5′-thymidine residue or any combination thereof.

In some embodiments, the primer pair is configured to produce an amplification product of at least a portion of an STR locus selected from the group consisting of DYS393, DYS19, DYS391, DYS385a/b, DYS390, DYS392, DYS437, DYS438, DYS439, DYS389I and DYS389II.

In some embodiments, the locus from which the primer pair produces the amplification product is DYS393. In one aspect of this embodiment, each member of the primer pair has at least 70%, at least 80%, at least 90%, at least 95% or at least 100% sequence identity with a corresponding member of a primer pair selected from the group consisting of: SEQ ID NOs: 1:43, 63:54, 67:12, 62:64, 62:55, 33:31 and 34:30. In another aspect, each member of the primer pair has at least 70%, at least 80%, at least 90%, at least 95% or at least 100% sequence identity with a corresponding member of: SEQ ID NOs: 63:54. In a preferred aspect, the primer pair is the primer pair of SEQ ID NOs: 63:54.

In some embodiments, the locus from which the primer pair produces the amplification product is DYS19. In one aspect of this embodiment, each member of the primer pair has at least 70%, at least 80%, at least 90%, at least 95% or at least 100% sequence identity with a corresponding member of a primer pair selected from the group consisting of: SEQ ID NOs: 16:28, 51:17 and 45:60. In another aspect, each member of the primer pair has at least 70%, at least 80%, at least 90%, at least 95% or at least 100% sequence identity with a corresponding member of: SEQ ID NOs: 51:17. In a preferred aspect, the primer pair is the primer pair of SEQ ID NOs: 51:17.

In some embodiments, the locus from which the primer pair produces the amplification product is DYS391. In one aspect of this embodiment, each member of the primer pair has at least 70%, at least 80%, at least 90%, at least 95% or at least 100% sequence identity with a corresponding member of a primer pair selected from the group consisting of: SEQ ID NOs: 32:50, 19:13, 19:48, and 70:57. In another aspect, each member of the primer pair has at least 70%, at least 80%, at least 90%, at least 95% or at least 100% sequence identity with a corresponding member of: SEQ ID NOs: 19:48. In a preferred aspect, the primer pair is the primer pair of SEQ ID NOs: 19:48.

In some embodiments, the locus from which the primer pair produces the amplification product is DYS391. In one aspect of this embodiment, each member of the primer pair has at least 70%, at least 80%, at least 90%, at least 95% or at least 100% sequence identity with a corresponding member of a primer pair selected from the group consisting of: SEQ ID NOs: 10:27, 42:27, 10:35, 42:66 and 72:67. In another aspect, each member of the primer pair has at least 70%, at least 80%, at least 90%, at least 95% or at least 100% sequence identity with a corresponding member of: SEQ ID NOs: 72:67. In a preferred aspect, the primer pair is the primer pair of SEQ ID NOs: 72:67.

In some embodiments, the locus from which the primer pair produces the amplification product is DYS390. In one aspect of this embodiment, each member of the primer pair has at least 70%, at least 80%, at least 90%, at least 95% or at least 100% sequence identity with a corresponding member of a primer pair selected from the group consisting of: SEQ ID NOs: 59:20, 21:49, 59:49, 39:68 and 73:74. In another aspect, each member of the primer pair has at least 70%, at least 80%, at least 90%, at least 95% or at least 100% sequence identity with a corresponding member of: SEQ ID NOs: 39:68. In a preferred aspect, the primer pair is the primer pair of SEQ ID NOs: 39:68.

In some embodiments, the locus from which the primer pair produces the amplification product is DYS437. In one aspect of this embodiment, each member of the primer pair has at least 70%, at least 80%, at least 90%, at least 95% or at least 100% sequence identity with a corresponding member of a primer pair selected from the group consisting of: SEQ ID NOs: 65:44, 36:14, 8:14, 38:61, and 36:37. In another aspect, each member of the primer pair has at least 70%, at least 80%, at least 90%, at least 95% or at least 100% sequence identity with a corresponding member of: SEQ ID NOs: 36:37. In a preferred aspect, the primer pair is the primer pair of SEQ ID NOs: 36:37.

In some embodiments, the locus from which the primer pair produces the amplification product is DYS438. In one aspect of this embodiment, each member of the primer pair has at least 70%, at least 80%, at least 90%, at least 95% or at least 100% sequence identity with a corresponding member of a primer pair selected from the group consisting of: SEQ ID NOs: 7:56, 71:41, 22:6, and 71:9. In another aspect, each member of the primer pair has at least 70%, at least 80%, at least 90%, at least 95% or at least 100% sequence identity with a corresponding member of: SEQ ID NOs: 22:6. In a preferred aspect, the primer pair is the primer pair of SEQ ID NOs: 22:6.

In some embodiments, the locus from which the primer pair produces the amplification product is DYS439. In one aspect of this embodiment, each member of the primer pair has at least 70%, at least 80%, at least 90%, at least 95% or at least 100% sequence identity with a corresponding member of a primer pair selected from the group consisting of: SEQ ID NOs: 3:58, 2:40, 4:52, and 2:52. In another aspect, each member of the primer pair has at least 70%, at least 80%, at least 90%, at least 95% or at least 100% sequence identity with a corresponding member of: SEQ ID NOs: 4:52. In a preferred aspect, the primer pair is the primer pair of SEQ ID NOs: 4:52.

In some embodiments, the locus from which the primer pair produces the amplification product is DYS389I. In one aspect of this embodiment, each member of the primer pair has at least 70%, at least 80%, at least 90%, at least 95% or at least 100% sequence identity with a corresponding member of a primer pair selected from the group consisting of: SEQ ID NOs: 23:15, and 23:5. In another aspect, each member of the primer pair has at least 70%, at least 80%, at least 90%, at least 95% or at least 100sequence identity with a corresponding member of: SEQ ID NOs: 23:5. In a preferred aspect, the primer pair is the primer pair of SEQ ID NOs: 23:5.

In some embodiments, the locus from which the primer pair produces the amplification product is DYS389II. In one aspect of this embodiment, each member of the primer pair has at least 70%, at least 80%, at least 90%, at least 95% or at least 100% sequence identity with a corresponding member of SEQ ID NOs: 24:47. In a preferred aspect, the primer pair is the primer pair of SEQ ID NOs: 24:47.

Another aspect is a kit which includes one or more purified oligonucleotide primer pairs for identifying a known STR allele or characterizing a previously unknown STR allele in a nucleic acid sample. The one or more primer pairs is configured to produce an amplification product of an STR locus. The amplification product duplicates the sequence of the known STR allele or the previously unknown STR allele. Each member of the one or more primer pairs has at least 70%, at least 80%, at least 90%, at least 95% or at least 100% sequence identity with a corresponding member of one or more primer pairs selected from the group consisting of: SEQ ID NOs: 16:28, 51:17, 45:60, 10:27, 42:27, 10:35, 24:46, 23:15, 23:5, 24:47, 59:20, 21:49, 59:49, 39:68, 32:50, 19:13, 19:48, 70:57, 26:11, 53:29, 25:18, 69:18, 1:43, 63:54, 67:12, 62:64, 65:44, 36:14, 8:14, 38:61, 36:37, 7:56, 71:41, 22:6, 71:9, 3:58, 2:40, 4:52, 2:52, 62:55, 33:31, 34:30, 73:74, 42:66 and 72:67.

In one embodiment of the kit, one or more primer pairs are contained within the same reaction vessel, preferably a well of a 96-well plate. In some embodiments, the well includes five primer pairs and each member of the primer pairs has at least 70%, at least 80%, at least 90%, at least 95% or at least 100% sequence identity with a corresponding member of SEQ ID NOs: 23:5, 53:29, 19:48, 63:54 and 39:68. This kit may further include at least a first additional well which includes four primer pairs and each member of the primer pairs has at least 70%, at least 80%, at least 90%, at least 95% or at least 100% sequence identity with a corresponding member of SEQ ID NOs: 24:47, 22:6, 4:52 and 36:37. This kit may further include at least a second additional well comprising an additional primer pair. Each member of this additional primer pair has at least 70%, at least 80%, at least 90%, at least 95% or at least 100% sequence identity with a corresponding member of SEQ ID NOs: 51:17. This kit may further include at least a third additional well comprising a primer pair. Each member of this primer pair has at least 70%, at least 80%, at least 90%, at least 95% or at least 100% sequence identity with a corresponding member of SEQ ID NOs: 72:67.

In some embodiments, the kit includes deoxynucleotide triphosphates comprising: 13C-enriched dGTP, dTTP, dCTP and/or dATP. In an additional embodiment, the kits and methods described herein include or use all of the components to perform polymerase chain reaction (PCR). These components include, but are not limited to, deoxynucleotide triphosphates (dNTPs) for each nucleobase, a thermostable DNA polymerase and buffers useful in performing PCR.

In another embodiment, there is provided a method of identifying an individual. A DNA-containing sample is obtained from the individual and a plurality of STR alleles of the DNA is identified according to the methods described above. The plurality of STR alleles provides an allelic profile for the individual. The allelic profile of the individual is then compared with a plurality of database-stored allelic profiles of known individuals. A match between the allelic profile and a member of the plurality of database-stored allelic profiles identifies the individual. In some embodiments, a plurality of amplification products is produced in the same reaction vessel, preferably a 96-well plate.

In some embodiments of method of identifying an individual, the plurality of amplification products comprises five amplification products produced with five primer pairs. Preferably, each member of the five primer pairs has at least 70%, at least 80%, at least 90%, at least 95% or at least 100% sequence identity with a corresponding member of SEQ ID NOs: 23:5, 53:29, 19:48, 63:54 and 39:68. In an additional embodiment, the method includes producing four additional amplification products in at least one additional reaction vessel. The four additional amplification products are produced with four primer pairs. Preferably, each member of the four primer pairs has at least 70%, at least 80%, at least 90%, at least 95% or at least 100% sequence identity with a corresponding member of SEQ ID NOs: 24:47, 22:6, 4:52, and 36:37. In an additional embodiment, the method includes producing two additional amplification products in separate reaction vessels with two primer pairs. Preferably, each member of the two primer pairs has at least 70%, at least 80%, at least 90%, at least 95% or at least 100% sequence identity with a corresponding member of SEQ ID NOs: 51:17 and 72:67.

In another embodiment, a system is provided which includes a mass spectrometer configured to detect one or more molecular masses of amplicons produced using at least one purified oligonucleotide primer pair that comprises forward and reverse primers. The forward and reverse primers comprise nucleic acid sequences independently having at least 70%, at least 80%, at least 90%, at least 95% or at least 100% sequence identity with a corresponding member of a primer pair selected from the group consisting of: SEQ ID NOs: 16:28, 51:17, 45:60, 10:27, 42:27, 10:35, 24:46, 23:15, 23:5, 24:47, 59:20, 21:49, 59:49, 39:68, 32:50, 19:13, 19:48, 70:57, 26:11, 53:29, 25:18, 69:18, 1:43, 63:54, 67:12, 62:64, 65:44, 36:14, 8:14, 38:61, 36:37, 7:56, 71:41, 22:6, 71:9, 3:58, 2:40, 4:52, 2:52, 62:55, 33:31, 34:30, 73:74, 42:66 and 72:67. The system further includes a controller operably connected to the mass spectrometer. The controller is configured to correlate the molecular masses of the amplicons with an identity of a known STR allele. The controller is further configured to characterize a previously unknown molecular mass as representing a previously unknown STR allele.

In some embodiments, the controller is configured to determine base compositions of the amplicons from the molecular masses of the amplicons. The base compositions correspond to known STR alleles. In one aspect, the controller includes or is operably connected to a database of known molecular masses and/or known base compositions of amplicons of known STR alleles produced with the primer pair.

### BRIEF DESCRIPTION OF THE DRAWINGS

FIG. 1 is a flow chart illustrating an example of a primer selection and STR-typing method provided herein.

FIG. 2 is a mass spectrum of an amplification product of SeraCare sample SC35495 obtained with primer pair number 4582.

FIG. 3A is a mass spectrum of an amplification product of SeraCare sample SC35495 obtained with primer pair number 4586.

FIG. 3B is a mass spectrum of an amplification product of SeraCare sample SC35495 obtained with primer pair number 4587.

FIG. 4 is a mass spectrum of a pair of amplification products amplified from SeraCare sample SC35495 obtained with primer pair number 4602. One amplification product has a T→C SNP relative to the other product.

FIG. 5 is a mass spectrum obtained from a multiplex (5-plex) amplification reaction of SeraCare sample SC35495 using primer pair numbers 4586, 4591, 4594, 4597, and 4602.

FIG. 6 is a mass spectrum obtained from a multiplex (4-plex) amplification reaction of SeraCare sample SC35495 using primer pair numbers 4587, 4608, 4611 and 4615.

FIG. 7 is a mass spectrum obtained from a multiplex amplification reaction of NIST sample WT51378 using primer pair numbers 4587, 4608, 4611 and 4615.

FIG. 8 is an expanded region of the mass spectrum of FIG. 7 showing mass spectral signals of the two strands of the DYS438 amplification product obtained with primer pair number 4611.

FIG. 9 is an alignment of the sequences of expected amplification products for the nine known alleles of the DYS438 locus. Primer hybridization coordinates are also indicated.

### DESCRIPTION OF EMBODIMENTS

As used herein a “sample” refers to anything capable of being analyzed by the methods provided herein. In preferred embodiments, the sample comprises or is suspected one or more nucleic acids capable of analysis by the methods. Preferably, the samples comprise DNA. Samples can be forensic samples, which can include, for example, evidence from a crime scene, blood, blood stains, semen, semen stains, bone, teeth, hair saliva, urine, feces, fingernails, muscle tissue, cigarettes, stamps, envelopes, dandruff, fingerprints, and personal items. In some embodiments, me samples are mixture samples, when comprise nucleic acids from more than one subject or individual. In some embodiments, the methods provided herein comprise purifying the sample or purifying the nucleic acid(s) from the sample. In some embodiments, the sample is purified nucleic acid or DNA.

As used herein, “repeated DNA sequence,” “tandem repeat locus,” “tandem DNA repeat” and “satellite DNA” refer to repeated DNA sequences present in eukaryotic genomes. “VNTRs” (variable nucleotide tandem repeats) or “minisatellites” refer to medium sized repeat units that are about 10-100 linked nucleotides in length. The terms “short tandem repeat,” “STR”, “simple sequence repeats” “SSR” and “microsatellite” refer to tandem DNA repeat regions having core units of between 2-6 nucleotides in length. STRs are characterized by the number of nucleotides in the core repeat unit. Dinucleotide, trinucleotide, and tetranucleotide STRs represent STRs with core repeat units of 2, 3, and 4 respectively.

The term “STR locus,” (also known as “STR marker”) refers to a particular place on a chromosome where the region of short tandem repeats is located. Particular sequence variations (number of repeat units and sequence polymorphisms) found at an STR locus are called “STR alleles.” There are often several STR alleles for one STR locus within any given population. An individual can have more than one STR allele (one on each chromosome—maternal and paternal) for a given STR locus. Such an individual is said to be “heterozygous” at the particular STR locus. Individual variations of such loci are called alleles. An individual with identical alleles on both chromosomes is said to be “homozygous.” It is notable that, in context of Y-STRs (STRs located on the human Y chromosome which is found only in males) each human male will carry only one instance of the STR locus and therefore, characterization as homozygous or heterozygous is not applicable. For a particular STR locus, individuals in a population differ in the number of these core repeat units. Alleles at a particular STR locus can be said to be corresponding to that STR locus.

As used herein, “same-length STR alleles” or “same-length alleles” are used to refer to two or more alleles that share a common number of linked nucleotides or sequence length at the STR locus. Same-length alleles can differ in base composition or sequence. “Sequence length” refers to the number of linked nucleotides for a given nucleic acid, nucleic acid sequence or portion or region of such a sequence.

For certain STR loci, microvariant alleles have been identified that differ from common allele variants by one or more base pairs. These variations can be in the form of nucleotide insertion, deletion or nucleotide base changes. One such variation, “single nucleotide polymorphism” or “SNP” refers to a single nucleotide change compared with a reference sequence or common sequence. In some embodiments, the methods provided herein can discriminate alleles based on one or more SNPs, and can identify SNPs in STR loci.

A common nomenclature for STR loci and STR alleles developed by the International Society of Forensic Haemogenetics (ISFH) (Bar et al. Int. J. Legal Med. 1997, 107, 159-160). Alleles are named based on number of the core repeat unit. For example, an allele designated 12 for a particular STR locus would have 12 repeat units. Incomplete repeat units are designated with a decimal point following the whole number, for example, 12.2.

As used herein, “forensic DNA typing” refers to forensic methods for determining a genotype of any one or more loci of an individual, nucleic acid, sample, or evidence. “STR-typing” refers to forensic DNA typing or DNA typing using methods to determine genotype of one or more STR loci. STR-typing can be used for such purposes as forensics, identity testing, paternity testing, and other human identification means. Often, STR typing involves the amplification of multiple STR DNA loci that display a collection of alleles in the human population that differ in repeat number for each locus examined.

As used herein, “conventional STR-typing” or “standard STR-typing” refer to the most common available methods used for STR typing. Specifically, the terms “conventional amplification-based STR typing” and “standard amplification-based STR typing” refer to the most common methods where STR loci are identified by amplification and resolved by assigning allele designations based on size or sequence length. Often, the products of such amplification reactions are analyzed by electrophoresis using fluorescent detection methods, and subsequent discrimination among different alleles based on amplification product length. The methods provided herein can be distinguished from conventional amplification based STR-typing. For example, the methods provided herein provide the ability to assign allele designations for STR loci based upon size as determined by mass. In addition, the methods provided herein can further resolve apparently homozygous alleles by deriving information from the loci nucleotide sequence as measured by mass or base composition uncovering additional alleles within the loci. “Allele call” in STR-typing refers to a genotype, STR-type or particular allele identified by a STR-typing method for an individual, nucleic acid or sample.

As used herein, “primers,” “primer pairs” or “oligonucleotide primer pairs” are oligonucleotides that are designed to hybridize to conserved sequence regions within target nucleic acids, wherein the conserved sequence regions are conserved among two or more nucleic acids, alleles, or individuals. A primer pair is a pair of primers and thus comprises a forward and a reverse primer. In some embodiments, the conserved sequence regions (and thus the hybridized primers) flank an intervening variable nucleic acid region that varies among two or more alleles or individuals. Upon amplification, the primer pairs yield amplification products (also called amplicons) that comprise base composition variability between two or more individuals or nucleic acids. The variability of the base compositions allows for the identification of one or more individuals or a genotype of one or more individuals based on the amplicons and their base composition distinctions. In a preferred embodiment, primer pairs are designed to hybridize to regions that are directly adjacent to or nearly adjacent to the STR locus. It will be apparent, however, that some variations of the primers provided herein will serve to provide effective amplification of desired sequences. Such variations could include, for example, adding or deleting one or a few bases from the primer and/or shifting the position of the primer relative to the STR locus or variable region.

In some embodiments of the invention, the oligonucleotide primer pairs described herein can be purified. As used herein, “purified oligonucleotide primer pair,” “purified primer pair,” or “purified” means an oligonucleotide primer pair that is chemically-synthesized to have a specific sequence and a specific number of linked nucleosides. This term is meant to explicitly exclude nucleotides that are generated at random to yield a mixture of several compounds of the same length each with randomly generated sequence.

The primer pairs are designed to generate amplicons that are amenable to molecular mass analysis. Standard primer pair nomenclature is used herein, and includes naming of a reference sequence, hybridization coordinates, and other identifying information. For example, the forward primer for primer pair number 4578 is named DYS19_AC017019_RC_{—}118941_{—}118971_F. The reference sequence for this primer (referred to in the name) is the reverse complement of Gen Bank Accession Number: AC017019. The number range “118941_{—}118971” indicates that the primer hybridizes to these nucleotide coordinates within the reference sequence. The “F” denotes that this particular primer is the forward primer of the pair. The “RC,” when present, indicates that the primer pair was designed using the reverse complement of the indicated GenBank sequence as the reference sequence. The beginning of the primer name refers to the locus, gene, or other nucleic acid region or feature to which the primer is targeted, and thus hybridizes within. The person skilled in the art will recognize that in order to design a primer pair which has a forward and a reverse primer which hybridize to opposite strands of a double stranded DNA in order to amplify the DNA, the forward primer is designed to hybridize to a sequence of a first strand while the reverse primer is designed to hybridize to the opposite strand. The information for designing the reverse primer is included in the first strand and is conveniently obtained by generating its “reverse complement.” Continuing with the example above, primer pair number 4578 has a forward primer (DYS19_AC017019-RC_{—}118941_{—}118971_F) which was designed to hybridize to a reference sequence represented by the reverse complement of GenBank Accession number AC017019 at a segment extending from position 118941 to 118971. Primer pair number 4578 has a reverse primer (DYS19_AC017019-RC_{—}119096_{—}119119_R) which is designed to hybridize to the reverse complement of the reference sequence at a segment extending from position 119096 to 119119. The primer names indicate that the primers are targeted to DYS19, a particular human STR locus. The primer pairs are selected and designed; however, to hybridize with two or more nucleic acids or nucleic acids from two or more individuals. So, the nomenclature used is merely to provide a reference sequence, and not to indicate that the primers hybridize with and generate an amplification product only from the reference sequence. Further, the sequences of the primer members of the primer pairs are not necessarily fully complementary to the conserved region of the reference sequence. Rather, the sequences are designed to be “best fit” amongst a plurality of nucleic acids at these conserved binding sequences. Therefore, the primer members of the primer pairs have substantial complementarity with the conserved regions of the nucleic acids, including the reference sequence nucleic acid.

As is used herein, the term “substantial complementarity means that a primer member or a primer pair comprises between about 70%-100%, or between about 80-100%, or between about 90-100%, or between about 95-100%, or between about 99-100% complementarity with the conserved binding sequence of a nucleic acid from an individual. Similarly, the primer pairs provided herein may comprise between about 70%-100%, or between about 80-100%, or between about 90-100%, or between about 95-100% identity, or between about 99-100% sequence identity with the primer pairs disclosed in Table 5 These ranges of complementarity and identity are inclusive of all whole or partial numbers embraced within the recited range numbers. For example, and not limitation, 75.667%, 82%, 91.2435% and 97% complementarity or sequence identity are all numbers that fall within the above recited range of 70% to 100%, therefore forming a part of this description. In some embodiments, any oligonucleotide primer pair may have one or both primers with less then 70% sequence homology with a corresponding member of any of the primer pairs of Table 5 if the primer pair has the capability of producing an amplification product corresponding to the desired STR-identifying amplicon.

In some embodiments, the oligonucleotide primers are 13 to 40 nucleobases in length (13 to 35 linked nucleotide residues). These embodiments comprise oligonucleotide primers 13, 14, 15, 16, 17, 18, 19, 20, 21, 22, 23, 24, 25, 26, 27, 28, 29, 30, 31, 32, 33, 34, 35, 36, 37, 38, 39 or 40 nucleobases in length, or any range therewithin. The present invention contemplates using both longer and shorter primers. Furthermore, the primers may also be linked to one or more other desired moieties, including, but not limited to, affinity groups, ligands, regions of nucleic acid that are not complementary to the nucleic acid to be amplified, labels, etc. In other embodiments, any oligonucleotide primer pair may have one or both primers with a length greater than 40 nucleobases if the primer pair has the capability of producing an amplification product corresponding to the desired STR-identifying amplicon.

As used herein, the term “variable region” is used to describe a region that, in some embodiments, falls between the conserved regions to which primer pairs described herein hybridize. The primers described herein can be designed such that, when hybridized to the target, they flank variable regions. Variable regions possess distinct base compositions between two or more individuals or alleles, such that at least two alleles, nucleic acids from at least two individuals, or at least two nucleic acids can be resolved from one another by determining the base composition of the amplicon generated by the primers that flank such a variable region when bound, or in other words bind to sequence regions that flank the variable region. In one embodiment, the variable region comprises an STR locus. In one aspect, the variable region comprises a distinct base composition among two or more amplicons generated from two distinct alleles that comprise the same number of nucleotides, and are thus the same length. In one aspect, the base composition of the variable region differs only in sequence, and not in length among two or more alleles.

As used herein, the term “amplicon” and “amplification product” refer to a nucleic acid generated or capable of generation using the primer pairs and methods described herein. In particular, “STR-identifying amplicons,” also called “STR-typing amplicons,” “STR-typing amplification products,” and “STR-identifying amplification products” are amplicons that can be used to determine the genotype (or identify the particular allele) for an individual nucleic acid at an STR locus. In some embodiments, the STR-typing amplicons are generated using in silico methods using electronic PCR and an electronic representation of primer pairs. The amplicons generated using in silico methods can be used to populate a database. The amplicon is preferably double stranded DNA; however, it can be RNA and/or DNA:RNA. The amplicon comprises the sequences of the conserved regions/primer pairs and the intervening variable region. As discussed herein, primer pairs are designed to generate amplicons from two or more alleles. The base composition of any given amplicon will include the primer pair, the complement of the primer pair, the conserved regions and the variable region from the nucleic acid that was amplified to generate the amplicon. One skilled in the art understands that the incorporation of the designed primer pair sequences into any amplicon will replace the native sequences at the primer binding site, and complement thereof. After amplification of the target region using the primers the resultant amplicons, including the primer sequences, generate the molecular mass data. Amplicons having any native sequences at the primer binding sites, or complement thereof, are undetectable because of their low abundance. Such is accounted for when identifying one or more nucleic acids from one or more alleles using any particular primer pair. The amplicon further comprises a length that is compatible with mass spectrometry analysis. STR-identifying amplicons (STR-typing amplicons) generate base composition signatures that are preferably unique to the identity of an STR allele.

Preferably, amplicons comprise from about 45 to about 200 consecutive nucleobases (i.e., from about 45 to about 200 linked nucleosides). One of ordinary skill in the art will appreciate that this range expressly embodies compounds of 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, and 200 nucleobases in length. One ordinarily skilled in the art will further appreciate that the above range is not an absolute limit to the length of an amplicon, but instead represents a preferred length range. Amplicons lengths falling outside of this range are also included herein so long as the amplicon is amenable to calculation of a base composition signature as herein described. As used herein, the term “about” means encompassing plus or minus 10%. For example, the term “about 200 nucleotides” refers to a range encompassing between 180 and 220 nucleotides.

As used herein, the term “molecular mass” refers to the mass of a compound as determined using mass spectrometry. Herein, the compound is preferably a nucleic acid, more preferably a double stranded nucleic acid, still more preferably a double stranded DNA nucleic acid and is most preferably an amplicon. When the nucleic acid is double stranded the molecular mass is determined for both strands. Here, the strands are separated either before introduction into the mass spectrometer, or the strands are separated by the mass spectrometer (for example, electro-spray ionization will separate the hybridized strands). The molecular mass of each strand is measured by the mass spectrometer.

As used herein, the term “base composition” refers to the number of each residue comprising an amplicon, without consideration for the linear arrangement of these residues in the strand(s) of the amplicon. The amplicon residues comprise, adenosine (A), guanosine (G), cytidine, (C), (deoxy)thymidine (T), uracil (U), inosine (I), nitroindoles such as 5-nitroindole or 3-nitropyrrole, dP or dK (Hill et al.), an acyclic nucleoside analog containing 5-nitroindazole (Van Aerschot et al., Nucleosides and Nucleotides, 1995, 14, 1053-1056), the purine analog 1-(2-deoxy-.beta.-D-ribofuranosyl)-imidazole-4-carboxamide, 2,6-diaminopurine, 5-propynyluracil, 5-propynylcytosine, phenoxazines, including G-clamp, 5-propynyl deoxy-cytidine, deoxy-thymidine nucleotides, 5-propynylcytidine, 5-propynyluridine and mass tag modified versions thereof, including 7-deaza-2′-deoxyadenosine-5-triphosphate, 5-iodo-2′-deoxyuridine-5′-triphosphate, 5-bromo-2′-deoxyuridine-5′-triphosphate, 5-bromo-2′-deoxycytidine triphosphate, 5-iodo-2′-deoxycytidine-5′-triphosphate, 5-hydroxy-2′-deoxyuridine-5′-triphosphate, 4-thiothymidine-5′-triphosphate, 5-aza-2′-deoxyuridine-5′-triphosphate, 5-fluoro-2′-deoxyuridine-5′-triphosphate, O6-methyl-2′-deoxyguanosine-5′-triphosphate, N2-methyl-2′-deoxyguanosine-5′-triphosphate, 8-oxo-2′-deoxyguanosine-5′-triphosphate or thiothymidine-5′-triphosphate. In some embodiments, the mass-modified nucleobase comprises 15.sup.N or 13.sup.C or both 15.sup.N and 13.sup.C. Preferably, the non-natural nucleosides used herein include 5-propynyluracil, 5-propynylcytosine and inosine. Herein the base composition for an unmodified DNA amplicon is notated as A.sub.wG.sub.xC.sub.yT.sub.z, wherein w, x, y and z are each independently a whole number representing the number of said nucleoside residues in an amplicon. Base compositions for amplicons comprising modified nucleosides are similarly notated to indicate the number of said natural and modified nucleosides in an amplicon. Base compositions are calculated from a molecular mass measurement of an amplicon, as described below. The calculated base composition for any given amplicon is then compared to a database of base compositions. In one embodiment, the database comprises base compositions of STR-typing amplicons. A match between the calculated base composition and a single database entry reveals the identity of the target nucleic acid or a genotype of an individual.

As is used herein, the term “base composition signature” refers to the base composition generated by any one particular amplicon.

As used herein, the term “database” is used to refer to a collection of base composition or molecular mass data. The base composition and/or molecular mass data in the database is indexed to specific individuals (subjects), alleles, or reference alleles and also to specific STR-identifying amplicons and primer pairs. In one embodiment, the data are indexed to particular STR loci. As used herein, a “reference allele” is an allele comprised in a database that has been previously determined to have a certain base composition, length, molecular mass, size and/or genotype. The reference allele may be indexed to primer pairs and amplicons provided herein. The base composition data reported in the database comprises the number of each nucleoside in an amplicon that would be generated for each allele or individual using each primer. The database can be populated by empirical data. In this aspect of populating the database, a nucleic acid with a particular allele or from a particular individual is selected and a primer pair is used to generate an amplicon. The molecular mass of the amplicon is determined using a mass spectrometer and the base composition calculated therefrom. An entry in the database is made to associate the base composition with the allele or individual and the primer pair used. The database may also be populated using other databases comprising allele or individual nucleic acid information. For example, using the GenBank database it is possible to perform electronic PCR using an electronic representation of a primer pair. Databases can be populated from other databases, such as FBI databases. This in silico method will provide the base composition for any or all selected allele(s) and/or individuals stored in the database. The information is then used to populate the base composition database as described above. A base composition database can be in silico, a written table, a reference book, a spreadsheet or any form generally amenable to databases. Preferably, it is in silico.

As used herein, the term “nucleobase” is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP). As is used herein, a nucleobase includes natural and modified residues, as described herein.

As used herein, a “wobble base” is a variation in a codon found at the third nucleotide position of a DNA triplet. Variations in conserved regions of sequence are often found at the third nucleotide position due to redundancy in the amino acid code.

The terms “homology,” “homologous” and “sequence identity” refer to a degree of identity. There may be partial homology or complete homology. A partially homologous sequence is one that is less than 100% identical to another sequence. Determination of sequence identity is described in the following example: a primer 20 nucleobases in length which is otherwise identical to another 20 nucleobase primer but having two non-identical residues has 18 of 20 identical residues (18/20=0.9 or 90% sequence identity). In another example, a primer 15 nucleobases in length having all residues identical to a 15 nucleobase segment of a primer 20 nucleobases in length would have 15/20=0.75 or 75% sequence identity with the 20 nucleobase primer. In context of the present invention, sequence identity is meant to be properly determined when the query sequence and the subject sequence are both described and aligned in the 5′ to 3′ direction. Sequence alignment algorithms such as BLAST, will return results in two different alignment orientations. In the Plus/Plus orientation, both the query sequence and the subject sequence are aligned in the 5′ to 3′ direction. On the other hand, in the Plus/Minus orientation, the query sequence is in the 5′ to 3′ direction while the subject sequence is in the 3′ to 5′ direction. It should be understood that with respect to the primers of the present invention, sequence identity is properly determined when the alignment is designated as Plus/Plus. Sequence identity may also encompass alternate or “modified” nucleobases that perform in a functionally similar manner to the regular nucleobases adenine, thymine, guanine and cytosine with respect to hybridization and primer extension in amplification reactions. In a non-limiting example, if the 5-propynyl pyrimidines propyne C and/or propyne T replace one or more C or T residues in one primer which is otherwise identical to another primer in sequence and length, the two primers will have 100% sequence identity with each other. In another non-limiting example, Inosine (I) may be used as a replacement for G or T and effectively hybridize to C, A or U (uracil). Thus, if inosine replaces one or more G or T residues in one primer which is otherwise identical to another primer in sequence and length, the two primers will have 100% sequence identity with each other. Other such modified or universal bases may exist which would perform in a functionally similar manner for hybridization and amplification reactions and will be understood to fall within this definition of sequence identity.

As used herein, “triangulation identification” means the employment of more than one primer pair, two or more primer pairs, three or more primer pairs, or a plurality of primer pairs to generate amplicons necessary for the identification or typing of a nucleic acid or individual. The more than one primer pair can be used in individual wells or in a multiplex PCR assay. In a “multiplex” assay, the methods provided herein are performed with two or more primer pairs simultaneously. Alternatively, a PCR reaction may be carried out in single wells comprising a different primer pair in each well. Following amplification the amplicons are pooled into a single well or container which is then subjected to molecular mass analysis. The combination of pooled amplicons can be chosen such that the expected ranges of molecular masses of individual amplicons are not overlapping and thus will not complicate identification of signals. Triangulation works as a process of elimination, wherein a first primer pair identifies that an unknown allele may be one of a group of alleles. Subsequent primer pairs are used in triangulation identification to further refine the identity of the allele amongst the subset of possibilities generated with the earlier primer pair. Triangulation identification is complete when the identity of the allele is determined. The triangulation identification process is also used to reduce false negative and false positive signals. Alternatively, if more than one primer pair are used in a multiplex assay, the combination of amplicons are generated simultaneously and can be analyzed simultaneously, comparing the multiple resultant molecular masses or base compositions to multiple amplicons in a database that are indexed to the different primer pairs used in the multiplex assay.

Provided herein are methods and compositions directed to unbiased forensic analysis and identity testing including STR typing of samples comprising nucleic acids using amplicons and ESI-MS to determine mass and base composition. The methods herein provide substantial accuracy to yield an unambiguous base composition (i.e. the number of A's, G's, C's and T's) which in turn can be used to derive a DNA profile for an individual. Importantly, these base composition profiles can be referenced to existing forensics databases derived from STR or other forensic marker profiles and/or can be added to such databases. Because the methods use molecular mass and base compositions to derive specific alleles, the methods and compositions provided herein are capable of detecting SNPs within STR regions that go undetected by conventional electrophoretic STR-typing analyses. For example, all instances of “allele type 18” for the DYS389II STR locus are not equivalent. A particular individual may contain an A to G (A→G) SNP, which distinguishes this individual from individuals containing the normal allele type 13 (see for example, sample JT51471 in the first row of Table 9A). Such an example of a SNP within an STR locus would not be expected to be detected by standard STR-typing methods and kits that use electrophoretic size discrimination to resolve STR alleles.

In a preferred embodiment, the amplicons are STR-identifying amplicons or STR-identifying amplification products. In this embodiment, primers are selected to hybridize to conserved sequence regions of nucleic acids, which flank a variable nucleic acid sequence region, derived from the samples to yield an STR-typing amplicon that can be amplified and is amenable to molecular mass determination. A base composition is calculated from the molecular mass, which indicates the number of each nucleotide in the amplicon. The molecular mass or corresponding base composition or base composition signature of the amplicon is then compared to a database comprising molecular masses or base composition signatures that are indexed to alleles and/or individuals and the primer pair that was used to generate the amplicon. A match of the determined molecular mass or calculated base composition to a molecular mass or base composition in the database associates the nucleic acid from the sample with an allele or individual indexed in the database. In some cases, the nucleic acid from the sample or a particular allele associates with more than one individual or identity. In these cases, one or more additional primer pairs are used either subsequently or simultaneously to generate one or more additional amplicons. The mass and base composition of the one or more additional amplicons are determined/calculated and the methods provided herein are used to compare the results to a database and further characterize and preferably identity the sample. This type of analysis can be carried out as described herein using triangulation, or using multiplex assays. The present method provides rapid throughput analysis and does not require nucleic acid sequencing for identification of nucleic acids from samples.

In one embodiment, the method is carried out with two or more primer pairs in a multiplex reaction. In one aspect, when the method is carried out in a multiplex reaction, it may be advantageous to use PCR reagents with high magnesium concentrations, for example, 3 mM magnesium chloride. As is known in the art, such reagents favor adenylation of amplification products. In one embodiment, it is advantageous to minimize split-peak results that can occur when there is adenylation of only a fraction of the amplification products in the sample, for example, generation of a fraction of the amplification products with a slightly different length than other products. Thus, in a preferred aspect, it is desired to promote full or about full adenylation. In one aspect, the primer pairs are configured so as to promote full adenylation such that one or both of the forward and reverse primer comprises a C or a G nucleobase at the 5′ end. Temperatures in the cycle reaction may also be adjusted to promote full adenylation while retaining efficacy, for example, by using an annealing temperature of about 61 degrees C.

In some embodiments, amplicons amenable to molecular mass determination which are produced by the primers described herein are either of a length, size or mass compatible with the particular mode of molecular mass determination or compatible with a means of providing a predictable fragmentation pattern in order to obtain predictable fragments of a length compatible with the particular mode of molecular mass determination. Such means of providing a predictable fragmentation pattern of an amplicon include, but are not limited to, cleavage with restriction enzymes or cleavage primers, for example. Thus, in some embodiments, amplicons are larger than 200 nucleobases and are amenable to molecular mass determination following restriction digestion. Methods of using restriction enzymes and cleavage primers are well known to those with ordinary skill in the art.

In some embodiments, amplicons are obtained using the polymerase chain reaction (PCR) which is a routine method to those with ordinary skill in the molecular biology arts. In some embodiments, the PCR is accomplished by using the polymerase chain reaction and a polymerase chain reaction is catalyzed by a polymerase enzyme whose function is modified relative to a native polymerase. In some embodiments the modified polymerase enzyme is exo(−) Pfu polymerase which catalyzes the addition of nucleotide residues to staggered restriction digest products to convert the staggered digest products to blunt-ended digest products. Other amplification methods may be used such as ligase chain reaction (LCR), low-stringency single primer PCR, and multiple strand displacement amplification (SDA). These methods are also known to those with ordinary skill. (Michael, S F., Biotechniques 1994, 16, 411-412 and Dean et al., Proc. Natl. Acad. Sci. U.S.A. 2002, 99, 5261-5266).

Mass spectrometry (MS)-based detection of PCR products provides a means for determination of BCS which has several advantages. MS is intrinsically a parallel detection scheme without the need for radioactive or fluorescent labels, since every amplification product is identified by its molecular mass. The current state of the art in mass spectrometry is such that less than femtomole quantities of material can be readily analyzed to afford information about the molecular contents of the sample. An accurate assessment of the molecular mass of the material can be quickly obtained, irrespective of whether the molecular weight of the sample is several hundred, or in excess of one hundred thousand atomic mass units (amu) or Daltons. Intact molecular ions can be generated from amplification products using one of a variety of ionization techniques to convert the sample to gas phase. These ionization methods include, but are not limited to, electrospray ionization (ES), matrix-assisted laser desorption ionization (MALDI) and fast atom bombardment (FAB). For example, MALDI of nucleic acids, along with examples of matrices for use in MALDI of nucleic acids, are described in WO 98/54751. The accurate measurement of molecular mass for large DNAs is limited by the adduction of cations from the PCR reaction to each strand, resolution of the isotopic peaks from natural abundance .sup.13C and .sup.15N isotopes, and assignment of the charge state for any ion. The cations are removed by in-line dialysis using a flow-through chip that brings the solution containing the PCR products into contact with a solution containing ammonium acetate in the presence of an electric field gradient orthogonal to the flow. The latter two problems are addressed by operating with a resolving power of >100,000 and by incorporating isotopically depleted nucleotide triphosphates into the DNA. The resolving power of the instrument is also a consideration. At a resolving power of 10,000, the modeled signal from the [M-14H+].sup.14-charge state of an 84-mer PCR product is poorly characterized and assignment of the charge state or exact mass is impossible. At a resolving power of 33,000, the peaks from the individual isotopic components are visible. At a resolving power of 100,000, the isotopic peaks are resolved to the baseline and assignment of the charge state for the ion is straightforward. The [.sup.13C, .sup.15N]-depleted triphosphates are obtained, for example, by growing microorganisms on depleted media and harvesting the nucleotides (Batey et al., Nucl. Acids Res., 1992, 20, 4515-4523).

While mass measurements of intact nucleic acid regions are believed to be adequate, tandem mass spectrometry (MS.sup.n) techniques may provide more definitive information pertaining to molecular identity or sequence. Tandem MS involves the coupled use of two or more stages of mass analysis where both the separation and detection steps are based on mass spectrometry. The first stage is used to select an ion or component of a sample from which further structural information is to be obtained. The selected ion is then fragmented using, e.g., blackbody irradiation, infrared multiphoton dissociation, or collisional activation. For example, ions generated by electrospray ionization (ESI) can be fragmented using IR multiphoton dissociation. This activation leads to dissociation of glycosidic bonds and the phosphate backbone, producing two series of fragment ions, called the w-series (having an intact 3′ terminus and a 5′ phosphate following internal cleavage) and the a-Base series (having an intact 5′ terminus and a 3′ furan).

The second stage of mass analysis is then used to detect and measure the mass of these resulting fragments of product ions. Such ion selection followed by fragmentation routines can be performed multiple times so as to essentially completely dissect the molecular sequence of a sample.

If there are two or more targets of similar molecular mass, or if a single amplification reaction results in a product which has the same mass as two or more reference standards, they can be distinguished by using mass-modifying “tags.” Such an oligonucleotide is said to be mass-modified. In this embodiment, a nucleotide analog or “tag” is incorporated during amplification (e.g., a 5-(trifluoromethyl)deoxythymidine triphosphate) which has a different molecular weight than the unmodified base so as to improve distinction of masses. Such tags are described in, for example, WO 97/33000, which is incorporated herein by reference in its entirety. This further limits the number of possible base compositions consistent with any mass. For example, 5-(trifluoromethyl)deoxythymidine triphosphate can be used in place of dTTP in a separate nucleic acid amplification reaction. Measurement of the mass shift between a conventional amplification product and the tagged product is used to quantitate the number of thymidine nucleotides in each of the single strands. Because the strands are complementary, the number of adenosine nucleotides in each strand is also determined.

In contrast the mass tag approach, in a preferred embodiment mass-modified dNTPs are employed to further limit the number of base pair combinations and also to resolve SNPs that are not resolvable when using unmodified dNTPs.

In another amplification reaction, the number of G and C residues in each strand is determined using, for example, the cytidine analog 5-methylcytosine (5-meC) or 5-prolynylcytosine (propyne C). The combination of the A/T reaction and G/C reaction, followed by molecular weight determination, provides a unique base composition. This method is summarized in Table 1.

TABLE 1

| | | | | | | Total | Total |
|---|---|---|---|---|---|---|---|
| | | | Total | Base | Base | base | base |
| | Double | Single | mass | info | info | comp. | comp. |
| | strand | strand | this | this | other | Top | Bottom |
| Mass tag | sequence | Sequence | strand | strand | strand | strand | strand |
| T [symbol] mass | T*ACGT*ACGT* | T*ACGT*ACGT* | 3x | 3T | 3A | 3T | 3A |
| (T*-T) = x | AT*GCAT*GCA | | | | | 2A | 2T |
| | | | | | | 2C | 2G |
| | | | | | | 2G | 2C |
| | | AT*GCAT*GCA | 2x | 2T | 2A | | |
| C [symbol] mass | TAC*GTAC*GT | TAC*GTAC*GT | 2x | 2C | 2G | | |
| (C*-C) = y | ATGC*ATGC*A | | | | | | |
| | | ATGC*ATGC*A | 2x | 2C | 2G | | |

In the example shown in Table 1, the mass tag phosphorothioate A (A*) was used to distinguish a Bacillus anthracis cluster. The B. anthracis (A₁₄G₉C₁₄T₉) had an average MW of 14072.26, and the B. anthracis (AᵢA*₁₃G₉C₁₄T₉) had an average molecular weight of 14281.11 and the phosphorothioate A an average molecular weight of +16.06 as determined by ESI-TOF MS.

In another example, assume the measured molecular masses of each strand are 30,000.115 Da and 31,000.115 Da respectively, and the measured number of dT and dA residues are (30, 28) and (28, 30). If the molecular mass is accurate to 100 ppm, there are 7 possible combinations of dG+dC possible for each strand. However, if the measured molecular mass is accurate to 10 ppm, there are only 2 combinations of dG+dC, and at 1 ppm accuracy there is only one possible base composition for each strand.

Signals from the mass spectrometer may be input to a maximum-likelihood detection and classification algorithm such as is widely used in radar signal processing. Processing may end with a Bayesian classifier using log likelihood ratios developed from the observed signals and average background levels. Background signal strengths are estimated and used along with the matched filters to form signatures which are then subtracted. The maximum likelihood process is applied to this “cleaned up” data in a similar manner employing matched filters and a running-sum estimate of the noise-covariance for the cleaned up data.

In some embodiments, the DNA analyzed is human DNA obtained from forensic samples, for example, human saliva, hair, blood, or nail.

Embodiments provided herein comprise primer pairs which are designed to bind to highly conserved sequence regions of DNA. In some embodiments, the conserved sequence regions flank an intervening variable region such as the variable sections found within regions STRs and yield amplification products which ideally provide enough variability to provide a forensic conclusion, and which are amenable to molecular mass analysis. By the term “highly conserved,” it is meant that the sequence regions exhibit from about 80 to 100%, or from about 90 to 100%, or from about 95 to 100% identity, or from about 80 to 99%, or from about 90 to 99%, or from about 95 to 99% identity. The molecular mass of a given amplification product provides a means of drawing a forensic conclusion due to the variability of the variable region. Thus, design of primers involves selection of a variable section with optimal variability in the DNA of different individuals.

The primer pairs are configured to produce an amplification product of an STR locus. The amplification product duplicates the sequence of the known STR allele or the previously unknown STR allele. Each member of the one or more primer pairs has at least 70%, at least 80%, at least 90%, at least 95% or at least 100% sequence identity with a corresponding member of one or more primer pairs selected from the group consisting of: SEQ ID NOs: 16:28, 51:17, 45:60, 10:27, 42:27, 10:35, 24:46, 23:15, 23:5, 24:47, 59:20, 21:49, 59:49, 39:68, 32:50, 19:13, 19:48, 70:57, 26:11, 53:29, 25:18, 69:18, 1:43, 63:54, 67:12, 62:64, 65:44, 36:14, 8:14, 38:61, 36:37, 7:56, 71:41, 22:6, 71:9, 3:58, 2:40, 4:52, 2:52, 62:55, 33:31, 34:30, 73:74, 42:66 and 72:67. In some embodiments, the STR locus is a Y-STR locus (located on a human Y chromosome).

In some embodiments, the conserved sequence region of DNA to which the primer pairs hybridize flank STR loci. Preferably, the STR loci are in a group of core “DYS” loci which include but are not limited to DYS393, DYS19, DYS391, DYS385a/b, DYS390, DYS392, DYS437, DYS438, DYS439, DYS389I, and DYS389II.

In one embodiment, the STR locus comprises DYS393. In one aspect each member of the primer pair has at least 70%, at least 80%, at least 90%, at least 95% or at least 100% sequence identity with the sequence of the corresponding member of a primer pair represented by any one or more of the following SEQ ID NOs: 1:43, 63:54, 67:12, 62:64, 62:55, 33:31 and 34:30.

In one embodiment, the STR locus comprises DYS19. In one aspect each member of the primer pair has at least 70%, at least 80%, at least 90%, at least 95% or at least 100% sequence identity with the sequence of the corresponding member of a primer pair represented by any one or more of the following SEQ ID NOs: 16:28, 51:17 and 45:60.

In one embodiment, the STR locus comprises DYS391. In one aspect each member of the primer pair has at least 70%, at least 80%, at least 90%, at least 95% or at least 100% sequence identity with the sequence of the corresponding member of a primer pair represented by any one or more of the following SEQ ID NOs: 32:50, 19:13, 19:48, and 70:57.

In one embodiment, the STR locus comprises DYS385a/b. In one aspect each member of the primer pair has at least 70%, at least 80%, at least 90%, at least 95% or at least 100% sequence identit with the sequence of the corresponding member of a primer pair represented by any one or more of the following SEQ ID NOs: 10:27, 42:27, 10:35, 42:66 and 72:67.

In one embodiment, the STR locus comprises DYS390. In one aspect each member of the primer pair has at least 70%, at least 80%, at least 90%, at least 95% or at least 100% sequence identity with the sequence of the corresponding member of a primer pair represented by any one or more of the following SEQ ID NOs: 59:20, 21:49, 59:49, 39:68 and 73:74.

In one embodiment, the STR locus comprises DYS392. In one aspect each member of the primer pair has at least 70%, at least 80%, at least 90%, at least 95% or at least 100% sequence identity with the sequence of the corresponding member of a primer pair represented by any one or more of the following SEQ ID NOs: 26:11, 53:29, 25:18, and 69:18.

In one embodiment, the STR locus comprises DYS437. In one aspect each member of the primer pair has at least 70%, at least 80%, at least 90%, at least 95% or at least 100% sequence identit with the sequence of the corresponding member of a primer pair represented by any one or more of the following SEQ ID NOs: 65:44, 36:14, 8:14, 38:61, and 36:37.

In one embodiment, the STR locus comprises DYS438. In one aspect each member of the primer pair has at least 70%, at least 80%, at least 90%, at least 95% or at least 100% sequence identity with the sequence of the corresponding member of a primer pair represented by any one or more of the following SEQ ID NOs: 7:56, 71:41, 22:6, and 71:9.

In one embodiment, the STR locus comprises DYS439. In one aspect each member of the primer pair has at least 70%, at least 80%, at least 90%, at least 95% or at least 100% sequence identity with the sequence of the corresponding member of a primer pair represented by any one or more of the following SEQ ID NOs: 3:58, 2:40, 4:52, and 2:52.

In one embodiment, the STR locus comprises DYS389I. In one aspect each member of the primer pair has at least 70%, at least 80%, at least 90%, at least 95% or at least 100% sequence identity with the sequence of the corresponding member of a primer pair represented by one or both of SEQ ID NOs: 23:15, and 23:5.

In one embodiment, the STR locus comprises DYS389II. In one aspect each member of the primer pair has at least 70%, at least 80%, at least 90%, at least 95% or at least 100% sequence identity with the sequence of the corresponding member of the primer pair represented by SEQ ID NOs: 24:47

In another embodiment, the primer pairs are combined and used in one or more multiplex reactions to generate an allelic profile for a sample obtained from an individual with the objective of identifying the individual. One aspect of this multiplex embodiment is configured to analyze 11 loci in four separate reactions comprising a five-plex reaction, a four-plex reaction and two single-plex reactions.

One aspect of this embodiment is configured, for example, with primer pairs targeting DYS389I, DYS392, DYS391, DYS393 and DYS390 in a five-plex reaction; primer pairs targeting DYS389II, DYS438, DYS439 and DYS437 in a four-plex reaction; a primer pair targeting DYS19 in a first single-plex reaction; and a primer pair targeting DYS385a/b in a second single-plex reaction. In this embodiment, 24 samples may be analyzed on a single 96-well plate which also includes four positive and four negative PCR control wells.

Ideally, primer hybridization sites are highly conserved in order to facilitate the hybridization of the primer. In cases where primer hybridization is less efficient due to lower levels of conservation of sequence, the primers provided herein can be chemically modified to improve the efficiency of hybridization. For example, because any variation (due to codon wobble in the 3^{rd} position) in these conserved regions among species is likely to occur in the third position of a DNA triplet, oligonucleotide primers can be designed such that the nucleotide corresponding to this position is a base which can bind to more than one nucleotide, referred to herein as a “universal base.” For example, under this “wobble” pairing, inosine (I) binds to U, C or A; guanine (G) binds to U or C, and uridine (U) binds to U or C. Other examples of universal bases include nitroindoles such as 5-nitroindole or 3-nitropyrrole (Loakes et al., Nucleosides and Nucleotides, 1995, 14, 1001-1003), the degenerate nucleotides dP or dK (Hill et al.), an acyclic nucleoside analog containing 5-nitroindazole (Van Aerschot et al., Nucleosides and Nucleotides, 1995, 14, 1053-1056) or the purine analog 1-(2-deoxy-beta-D-ribofuranosyl)-imidazole-4-carboxamide (Sala et al., Nucl. Acids Res., 1996, 24, 3302-3306).

In another embodiment, to compensate for the somewhat weaker binding by the “wobble” base, the oligonucleotide primers are designed such that the first and second positions of each triplet are occupied by nucleotide analogs which bind with greater affinity than the unmodified nucleotide. Examples of these analogs include, but are not limited to, 2,6-diaminopurine which binds to thymine, propyne T (5-propynyluridine) which binds to adenine and propyne C (5-propynylcytidine) and phenoxazines, including G-clamp, which binds to G. Propynylated pyrimidines are described in U.S. Pat. Nos. 5,645,985, 5,830,653 and 5,484,908, each of which is commonly owned and incorporated herein by reference in its entirety. Propynylated primers are claimed in U.S. Ser. No. 10/294,203 which is also commonly owned and incorporated herein by reference in entirety. Phenoxazines are described in U.S. Pat. Nos. 5,502,177, 5,763,588, and 6,005,096, each of which is incorporated herein by reference in its entirety. G-clamps are described in U.S. Pat. Nos. 6,007,992 and 6,028,183, each of which is incorporated herein by reference in its entirety. Thus, in other embodiments, the primer pair has at least one modified nucleobase such as 5-propynylcytidine or 5-propynyluridine.

Also provided herein are isolated DNA amplicons which are produced by the process of amplification of a sample of DNA with any of the above-mentioned primers.

While the methods compounds and compositions provided herein have been described with specificity in accordance with certain of its embodiments, the following examples serve only to illustrate the invention and are not intended to limit the same. The examples provided are only examples, and one skilled in the art will understand that other techniques can be used by those skilled in the art and such different techniques will not depart from the spirit of the invention (T. Maniatis et al., in Molecular Cloning. A. Laboratory Manual. CSH Lab. N.Y. (2001).

### EXAMPLES

### Example 1

### Nucleic Acid Isolation and Amplification

General Genomic DNA Sample Prep Protocol: Raw samples were filtered using Supor-200 0.2 μm membrane syringe filters (VWR International). Samples were transferred to 1.5 ml Eppendorf tubes pre-filled with 0.45 g of 0 7 mm Zirconia beads followed by the addition of 350 μl of ATL buffer (Qiagen, Valencia, Calif.). The samples were subjected to bead beating for 10 minutes at a frequency of 19 l/s in a Retsch Vibration Mill (Retsch). After centrifugation, samples were transferred to an S-block plate (Qiagen, Valencia, Calif.) and DNA isolation was completed with a BioRobot 8000 nucleic acid isolation robot (Qiagen, Valencia, Calif.).

Isolation of Blood DNA—Blood DNA was isolated using an MDx Biorobot according to according to the manufacturer's recommended procedure (Isolation of blood DNA on Qiagen QIAamp® DNA Blood BioRobot® MDx Kit, Qiagen, Valencia, Calif.). In some cases, DNA from blood punches were processed with a Qiagen QIAmp DNA mini kit using the manufacturer's suggested protocol for dried blood spots.

Isolation of Buccal Swab DNA—Since the manufacturer does not support a full robotic swab protocol, the blood DNA isolation protocol was employed after each swab was first suspended in 400 ml PBS+400 ml Qiagen AL buffer+20 μl Qiagen Protease solution in 14 ml round-bottom falcon tubes, which were then loaded into the tube holders on the MDx robot.

Isolation of DNA from Nails and Hairs—The following procedure employs a Qiagen DNeasy® tissue kit and represents a modification of the manufacturer's suggested procedure: hairs or nails were cut into small segments with sterile scissors or razorblades and placed in a centrifuge tube to which was added 1 ml of sonication wash buffer (10 mM TRIS-Cl, pH 8.0+10 mM EDTA+0.5% Tween-20. The solution was sonicated for 20 minutes to dislodge debris and then washed 2× with 1 ml ultrapure double deionized water before addition of 100 μl of Buffer X1 (10 mM TRIS-Cl, ph 8.0+10 mM EDTA+100 mM NaCl+40 mM DTT+2% SDS+250:g/ml Qiagen proteinase K). The sample was then incubated at 55° C. for 1-2 hours, after which 200 μl of Qiagen AL buffer and 210 μl isopropanol were solution was mixed by vortexing. The sample was then added to a Qiagen DNeasy mini spin column placed in a 2 ml collection tube and centrifuged for 1 min at 6000 g (8000 rpm). Collection tube and flow-through were discarded. The spin column was transferred to a new collection tube and 500 μl of buffer AW2 was added before centrifuging for 3 min. at 20,000 g (14,000 rpm) to dry the membrane. For elution, 50-100 μl of buffer AE was pipetted directly onto the DNeasy membrane and eluted by centrifugation (6000 g-8000 rpm) after incubation at room temperature for 1 min.

Amplification by PCR—An exemplary PCR procedure for amplification of DNA is the following: A 50 μl total volume reaction mixture contained 1× GenAmp® PCR buffer II (Applied Biosystems)—10 mM TRIS-Cl, pH 8.3 and 50 mM KCl, 1.5 mM MgCl₂, 400 mM betaine, 200 μM of each dNTP (Stratagene 200415), 250 nM of each primer, and 2.5-5 units of Pfu exo(−) polymerase Gold (Stratagene 600163) and at least 50 pg of template DNA. All PCR solution mixing was performed under a HEPA-filtered positive pressure PCR hood. An example of a programmable PCR cycling profile is as follows: 95° C. for 10 minutes, followed by 8 cycles of 95° C. for 20 sec, 62° C. for 20 sec, and 72° for 30 sec—wherein the 62° C. annealing step is decreased by 1° C. on each successive cycle of the 8 cycles, followed by 28 cycles of 95° C. for 20 sec, 55° C. for 20 sec, and 72° C. for 30 sec, followed by holding at 4° C. For multiplex reactions, in a preferred embodiment, PCR is carried out using 1 the Qiagen Multiplex PCR kit and buffers therein (Qiagen, Valencia, Calif.), which comprises 3 mM MgCl_{2.} 1 ng template DNA and 200 mM of each primer are used for a 40 μL reaction volume. The cycle conditions for an exemplary multiplex reaction are:
1- 95 degree C. 15 minutes
2- 95 degree C. 30 seconds
3- 61 degree C. 2 minutes (1-3 for 35 cycles)
4- 72 degree C. 30 seconds
5- 72 degree C. 10 minutes
6- 60 degree C. 30 minutes
7- 4 degree C. hold

Development and optimization of PCR reactions is routine to one with ordinary skill in the art and can be accomplished without undue experimentation.

### Example 2

### Purification of Amplification Products

Procedure for Semi-automated Purification of a PCR mixture using Commercially Available ZipTips®—As described by Jiang and Hofstadler (Y. Jiang and S. A. Hofstadler Anal. Biochem. 2003, 316, 50-57) an amplified nucleic acid mixture can be purified by commercially available pipette tips containing anion exchange resin. For pre-treatment of ZipTips® AX (Millipore Corp. Bedford, Mass.), the following steps were programmed to be performed by an Evolution™ P3 liquid handler (Perkin Elmer) with fluids being drawn from stock solutions in individual wells of a 96-well plate (Marshall Bioscience): loading of a rack of ZipTips®AX; washing of ZipTips®AX with 15 μl of 10% NH₄OH/50% methanol; washing of ZipTips® AX with 15 μl of water 8 times; washing of ZipTips® AX with 15 μl of 100 mM NH₄OAc.

For purification of a PCR mixture, 20 μl of crude PCR product was transferred to individual wells of a MJ Research plate using a BioHit (Helsinki, Finland) multichannel pipette. Individual wells of a 96-well plate were filled with 300 μl of 40 mM NH₄HCO₃. Individual wells of a 96-well plate were filled with 300 μl of 20% methanol. An MJ research plate was filled with 10 μl of 4% NH₄OH. Two reservoirs were filled with deionized water. All plates and reservoirs were placed on the deck of the Evolution P3 (EP3) (Perkin-Elmer, Boston, Mass.) pipetting station in pre-arranged order. The following steps were programmed to be performed by an Evolution P3 pipetting station: aspiration of 20 μl of air into the EP3 P50 head; loading of a pre-treated rack of ZipTips® AX into the EP3 P50 head; dispensation of the 20 μl NH₄HCO₃ from the ZipTips® AX; loading of the PCR product into the ZipTips® AX by aspiration/dispensation of the PCR solution 18 times; washing of the ZipTips® AX containing bound nucleic acids with 15 μl of 40 mM NH₄ HCO₃ 8 times; washing of the ZipTips® AX containing bound nucleic acids with 15 μl of 20% methanol 24 times; elution of the purified nucleic acids from the ZipTips® AX by aspiration/dispensation with 15 μl of 4% NH₄OH 18 times. For final preparation for analysis by ESI-MS, each sample was diluted 1:1 by volume with 70% methanol containing 50 mM piperidine and 50 mM imidazole.

Solution Capture Purification of PCR products for Mass Spectrometry with Ion-Exchange Resin-Magnetic Beads—The following procedure is disclosed in published U.S. Patent application US2005-0130196, filed on Sep. 17, 2004, which is commonly owned and incorporated herein by reference. For solution capture of nucleic acids with ion exchange resin linked to magnetic beads, 25 microliters of a 2.5 mg/mL suspension of BioClone amine-terminated supraparamagnetic beads are added to 25 to 50 microliters of a PCR or RT-PCR reaction containing approximately 10 pM of a typical PCR amplification product. The suspension is mixed for approximately 5 minutes by vortexing, pipetting or shaking, after which the liquid is removed following use of a magnetic separator to separate magnetic beads. The magnetic beads containing the amplification product are then washed 3 times with 50 mM ammonium bicarbonate/50% methanol or 100 mM ammonium bicarbonate/50% methanol, followed by three additional washes with 50% methanol. The bound PCR amplicon is eluted with electrospray-compatible elution buffer comprising 25 mM piperidine, 25 mM imidazole, 35% methanol, which can also comprise calibration standards. Steps of this procedure can be performed in multi-well plates and using a liquid handler, for example the Evolution™ P3 liquid handler and/or under the control of a robotic arm. The eluted nucleic acids in this condition are amenable to analysis by ESI-MS. The time required for purification of samples in a single 96-well plate using a liquid handler is approximately five minutes.

### Example 3

### Mass Spectrometry

The ESI-FTICR mass spectrometer used is a Bruker Daltonics (Billerica, Mass.) Apex II 70e electrospray ionization Fourier transform ion cyclotron resonance mass spectrometer (ESI-FTICR-MS) that employs an actively shielded 7 Tesla superconducting magnet. The active shielding constrains the majority of the fringing magnetic field from the superconducting magnet to a relatively small volume. Thus, components that might be adversely affected by stray magnetic fields, such as CRT monitors, robotic components, and other electronics can operate in close proximity to the ESI-FTICR mass spectrometer. All aspects of pulse sequence control and data acquisition are performed on a 1.1 GHz Pentium II data station miming Bruker's Xmass software. 20 μL sample aliquots are extracted directly from 96-well microtiter plates using a CTC HTS PAL autosampler (LEAP Technologies, Carrboro, N.C.) triggered by the data station. Samples are injected directly into the ESI source at a flow rate of 75 μL/hr. Ions are formed via electrospray ionization in a modified Analytica (Bradford Conn.) source employing an off axis, grounded electrospray probe positioned ca. 1.5 cm from the metalized terminus of a glass desolvation capillary. The atmospheric pressure end of the glass capillary is biased at 6000 V relative to the ESI needle during data acquisition. A counter-current flow of dry N₂/O₂ is employed to assist in the desolvation process. Ions are accumulated in an external ion reservoir comprised of an rf-only hexapole, a skimmer cone, and an auxiliary gate electrode, prior to injection into the trapped ion cell where they are mass analyzed.

Spectral acquisition is performed in the continuous duty cycle mode whereby ions are accumulated in the hexapole ion reservoir simultaneously with ion detection in the trapped ion cell. Following a 1.2 ms transfer event, in which ions are transferred to the trapped ion cell, the ions are subjected to a 1.6 ms chirp excitation corresponding to 8000-500 m/z. Data was acquired over an m/z range of 500-5000 (1M data points over a 225 K Hz bandwidth). Each spectrum is the result of co-adding 32 transients. Transients are zero-filled once prior to the magnitude mode Fourier transform and post calibration using the internal mass standard. The ICR-2LS software package (G. A. Anderson, J. E. Bruce (Pacific Northwest National Laboratory, Richland, Wash., 1995) is used to deconvolute the mass spectra and calculate the mass of the monoisotopic species using an “averaging” fitting routine (M. W. Senko, S. C. Beu, F. W. McLafferty, J. Am. Soc. Mass Spectrom. 1995, 6, 229) modified for DNA. Using this approach, monoisotopic molecular weights are calculated.

The ESI-TOF mass spectrometer used is based on a Bruker Daltonics MicroTOF™. Ions from the ESI source undergo orthogonal ion extraction and are focused in a reflectron prior to detection. The TOF is equipped with the same automated sample handling and fluidics as described for the FTICR above. Ions are formed in the standard MicroTOF™ ESI source that is equipped with the same off-axis sprayer and glass capillary as the FTICR ESI source. Consequently, source conditions are the same as those described above. External ion accumulation is also employed to improve ionization duty cycle during data acquisition. Each detection event on the TOF comprises 75,000 data points digitized over 75 μs.

The sample delivery scheme allows sample aliquots to be rapidly injected into the electrospray source at high flow rate and subsequently be electrosprayed at a much lower flow rate for improved ESI sensitivity. Prior to injecting a sample, a bolus of buffer is injected at a high flow rate to rinse the transfer line and spray needle to avoid sample contamination/carryover. Following the rinse step, the autosampler injects the next sample and the flow rate is switched to low flow. Following a brief equilibration delay, data acquisition begins. As spectra are co-added, the autosampler continues rinsing the syringe and picking up buffer to rinse the injector and sample transfer line. In general, two syringe rinses and one injector rinse are required to minimize sample carryover. During a routine screening protocol, a new sample mixture is injected every 106 seconds. A fast wash station for the syringe needle has also been implemented which, when combined with shorter acquisition times, facilitates the acquisition of mass spectra at a rate of just under one spectrum per minute.

Raw mass spectra are post-calibrated with an internal mass standard and deconvoluted to monoisotopic molecular masses. Unambiguous base compositions are derived from the exact mass measurement of the complementary single-stranded oligonucleotides. Quantitative results are obtained by comparing the peak heights with an internal PCR calibration standard present in every PCR well at 500 molecules per well. Calibration methods are commonly owned and disclosed in U.S. provisional patent Application Ser. No. 60/545,425, which is incorporated herein by reference in its entirety.

### Example 4

### De Novo Determination of Base Composition of Amplification Products Using Molecular Mass Modified Deoxynucleotide Triphosphates

Because the molecular masses of the four natural nucleotides have a relatively narrow molecular mass range (A=313.058, G=329.052, C=289.046, T=304.046—See Table 2), a persistent source of ambiguity in assignment of base composition can occur as follows: two nucleic acid strands having different base composition may have a difference of about 1 Da when the base composition difference between the two strands is G⇄A (−15.994) combined with C⇄T (+15.000). For example, one 99-mer nucleic acid strand having a base composition of A₂₇G₃₀C₂₁T₂₁ has a theoretical molecular mass of 30779.058 while another 99-mer nucleic acid strand having a base composition of A₂₆G₃₁C₂₂T₂₀ has a theoretical molecular mass of 30780.052. A 1 Da difference in molecular mass may be within the experimental error of a molecular mass measurement and thus, the relatively narrow molecular mass range of the four natural nucleotides imposes an uncertainty factor.

The present example provides for a means for removing this theoretical 1 Da uncertainty factor through amplification of a nucleic acid with one mass-tagged nucleotide and three natural nucleotides.

Addition of significant mass to one of the 4 nucleotides (dNTPs) in an amplification reaction, or in the primers themselves, will result in a significant difference in mass of the resulting amplification product (significantly greater than 1 Da) arising from ambiguities arising from the G⇄A combined with C⇄T event (Table 1). Thus, the same the G⇄A (−15.994) event combined with 5-Iodo-C⇄T (−110.900) event would result in a molecular mass difference of 126.894. If the molecular mass of the base composition A₂₇G₃₀5-Iodo-C₂₁T₂₁ (33422.958) is compared with A₂₆G₃₁5-Iodo-CT₂₀, (33549.852) the theoretical molecular mass difference is +126.894. The experimental error of a molecular mass measurement is not significant with regard to this molecular mass difference. Furthermore, the only base composition consistent with a measured molecular mass of the 99-mer nucleic acid is A₂₇G₃₀5-Iodo-C₂₁T₂₁. In contrast, the analogous amplification without the mass tag has 18 possible base compositions.

TABLE 2
Molecular Masses of Natural Nucleotides and the
Mass-Modified Nucleotide 5-Iodo-C and Molecular
Mass Differences Resulting from Transitions

| | Molecular | | |
|---|---|---|---|
| Nucleotide | Mass | Transition | Δ Molecular Mass |
| A | 313.058 | A-->T | −9.012 |
| A | 313.058 | A-->C | −24.012 |
| A | 313.058 | A-->5-Iodo-C | 101.888 |
| A | 313.058 | A-->G | 15.994 |
| T | 304.046 | T-->A | 9.012 |
| T | 304.046 | T-->C | −15.000 |
| T | 304.046 | T-->5-Iodo-C | 110.900 |
| T | 304.046 | T-->G | 25.006 |
| C | 289.046 | C-->A | 24.012 |
| C | 289.046 | C-->T | 15.000 |
| C | 289.046 | C-->G | 40.006 |
| 5-Iodo-C | 414.946 | 5-Iodo-C-->A | −101.888 |
| 5-Iodo-C | 414.946 | 5-Iodo-C-->T | −110.900 |
| 5-Iodo-C | 414.946 | 5-Iodo-C-->G | −85.894 |
| G | 329.052 | G-->A | −15.994 |
| G | 329.052 | G-->T | −25.006 |
| G | 329.052 | G-->C | −40.006 |
| G | 329.052 | G-->5-Iodo-C | 85.894 |

### Example 5

### Data Processing

Mass spectra of amplification products are analyzed independently using a maximum-likelihood processor, such as is widely used in radar signal processing, which is described in U.S. Patent Application 20040209260, which is incorporated herein by reference in entirety. This processor, referred to as GenX, first makes maximum likelihood estimates of the input to the mass spectrometer for each primer by running matched filters for each base composition aggregate on the input data. This includes the GenX response to a calibrant for each primer.

The algorithm emphasizes performance predictions culminating in probability-of-detection versus probability-of-false-alarm plots for conditions involving complex backgrounds of naturally occurring organisms and environmental contaminants Matched filters consist of a priori expectations of signal values given the set of primers used for each of the bioagents. A genomic sequence database is used to define the mass base count matched filters. The database contains the sequences of known bacterial bioagents and includes threat organisms as well as benign background organisms. The latter is used to estimate and subtract the spectral signature produced by the background organisms. A maximum likelihood detection of known background organisms is implemented using matched filters and a running-sum estimate of the noise covariance. Background signal strengths are estimated and used along with the matched filters to form signatures which are then subtracted. The maximum likelihood process is applied to this “cleaned up” data in a similar manner employing matched filters for the organisms and a running-sum estimate of the noise-covariance for the cleaned up data.

The amplitudes of all base compositions of bioagent identifying amplicons for each primer are calibrated and a final maximum likelihood amplitude estimate per organism is made based upon the multiple single primer estimates. Models of all system noise are factored into this two-stage maximum likelihood calculation. The processor reports the number of molecules of each base composition contained in the spectra. The quantity of amplification product corresponding to the appropriate primer set is reported as well as the quantities of primers remaining upon completion of the amplification reaction.

One of ordinary skill in the art will recognize that the signal processing methodologies of this example can be used in the context of the methods of STR analysis described herein.

### Example 6

### Amplification of Nucleic Acids With Isotope Depleted dNTPs

Due to the natural abundance of .sub.13C and other heavy isotopes in biological macromolecules, exact mass measurements are more difficult at increasing molecular weight. Additionally, the width of the isotopic distribution is inherently broader at high molecular weight thus making accurate monoisotopic molecular weight measurements difficult. There is also an inherent sensitivity loss as signals from a single amplicon are spread over more and more isotope peaks. An analogous problem occurs with ESI-MS analysis of proteins.

Isotope-depleted dNTPs suitable for use in PCR reactions can be produced from bacteria grown in isotope-depleted media in which the primary carbon source is .sub.13C depleted glucose and ¹⁵N depleted ammonium sulfate. Once the bacteria are grown to critical density, the isotope-depleted genomic DNA is extracted. DNA is then digested to mononucleotides from which deoxynucleotide triphosphates are enzymatically synthesized. In this manner, it should be possible to produce isotope-depleted reagents at modest cost. Proof-of-principle for this approach was recently published by Tang and coworkers (Tang et al., Anal. Chem., 2002, 74, 226-231). We expect that generating isotope depleted PCR products will result in a 3-5 fold improvement in sensitivity (as the signal is spread over fewer isotope peaks). More importantly, this approach should relieve the spectral congestion observed in the mass spectra and reduce the extent that species of similar mass or m/z produce overlapping MS peaks.

### Example 7

### Design of Primer Pairs for Development of a Forensic DNA Typing/Human Identity Assay

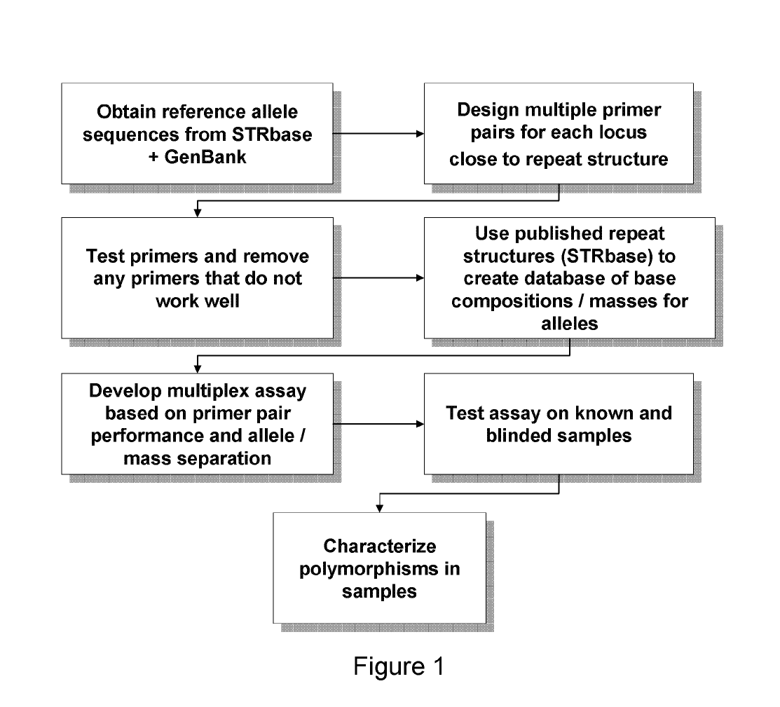

FIG. 1 is a flow diagram outlining the general approach for STR assay development, including primer design. In brief, reference allele sequences are obtained from the STR database or from GenBank. In most cases, two or more primer pairs are designed to hybridize at a region near an STR locus which is close to the repeat structure of the STR. These primer pairs are tested against samples containing an STR allele. Primers which do not produce a favorable yield of amplification products are discarded. The publically available STR database is used to develop a database of base compositions and masses of the expected amplification products for the known alleles. Commercially available software which performs PCR in silico may be used for this step. Once a panel of primers is chosen which produce good yields of amplification products, a multiplex scheme may be developed and used in testing known or blinded samples. This process may be used to characterize alleles which have SNPs relative to known alleles.

Primers were designed against each of the 11 core DYS loci according to the procedure outlined in this figure. Allele reference sequences were obtained for each STR locus from the STRbase database (Ruitberg, C. M.; Reeder, D. J.; Butler, J. M. Nucleic Acids Res. 2001, 29, 20-322). Multiple primers were designed for all but one STR locus. The multiple primers were designed to hybridize to conserved sequence regions adjacent or nearly adjacent (in close proximity) to the STR repeat. For example, Table 3 lists a series of named primers designed to hybridize within conserved regions flanking the core Y-STR loci. The sequences of these primers are provided in Table 5.

TABLE 3
Primer Pair Selection for the Core Y-STR Loci

| Primer | | |
|---|---|---|
| Pair Number | Primer Pair Name | Locus |
| 4578 | DYS19_AC017019-RC_118941_119119 | DYS19 |
| 4579 | DYS19_AC017019-RC | DYS19 |
| | _118947_119118 | |
| 4580 | DYS19_AC017019-RC | DYS19 |
| | _118947_119113 | |
| 4581 | DYS385-A-B_AC022486-RC | DYS385a/b |
| | _29394_29615 | |
| 4582 | DYS385-A-B-2_AC022486-RC | DYS385a/b |
| | _29491_29615 | |
| 4583 | DYS385-A-B-1_AC022486-RC | DYS385a/b |
| | _29394_29521 | |
| 4584 | DYS389I-II_AC004617-RC | DYS389I-II |
| | _125888_126106 | |
| 4585 | DYS389I_AC004617-RC | DYS389I |
| | _126008_126167 | |
| 4586 | DYS389I_AC004617-RC | DYS389I |
| | _126008_126107 | |
| 4587 | DYS389II-1_AC004617-RC | DYS389II-1 |
| | _125888_126021 | |
| 4588 | DYS390_AC011289_11029_11210 | DYS390 |
| 4589 | DYS390_AC011289_11022_11206 | DYS390 |
| 4590 | DYS390_AC011289_11029_11206 | DYS390 |
| 4591 | DYS390_AC011289_11034_11201 | DYS390 |
| 4592 | DYS391_G09613_18_181 | DYS391 |
| 4593 | DYS391_G09613_23_137 | DYS391 |
| 4594 | DYS391_G09613_23_142 | DYS391 |
| 4595 | DYS391_G09613_26_123 | DYS391 |
| 4596 | DYS392_AC011745-RC _97244_97358 | DYS392 |
| 4597 | DYS392_AC011745-RC _97256_97363 | DYS392 |
| 4598 | DYS392_AC011745-RC _97249_97362 | DYS392 |
| 4599 | DYS392_AC011745-RC _97237_97362 | DYS392 |
| 4600 | DYS393_AC006152_21087_21211 | DYS393 |
| 4601 | DYS393_AC006152_21089_21212 | DYS393 |
| 4602 | DYS393_AC006152_21090_21206 | DYS393 |
| 4603 | DYS393_AC006152_21092_21203 | DYS393 |
| 4604 | DYS437_AC002992_42957_43139 | DYS437 |
| 4605 | DYS437_AC002992_42956_43127 | DYS437 |
| 4606 | DYS437_AC002992_42951_43127 | DYS437 |
| 4607 | DYS437_AC002992_42949_43096 | DYS437 |
| 4608 | DYS437_AC002992_42956_43087 | DYS437 |
| 4609 | DYS438_AC002531_129796_129952 | DYS438 |
| 4610 | DYS438_AC002531_129798_129911_2 | DYS438 |
| 4611 | DYS438_AC002531_129788_129914 | DYS438 |
| 4612 | DYS438_AC002531_129798_129919_2 | DYS438 |
| 4613 | DYS439_AC002992_91258_91396 | DYS439 |
| 4614 | DYS439_AC002992_91262_91393 | DYS439 |
| 4615 | DYS439_AC002992_91254_91390 | DYS439 |
| 4616 | DYS439_AC002992_91262_91390 | DYS439 |
| 4670 | DYS393_AC006152_21092_21193 | DYS393 |
| 4671 | DYS393_AC006152_21092_21203_2 | DYS393 |
| 4672 | DYS393_AC006152_21089_21212_2 | DYS393 |
| 4673 | DYS390_AC011289_11034_11202 | DYS390 |
| 4691 | DYS385-A-B_AC022486-RC | DYS385a/b |
| | _29491_29634 | |
| 4692 | DYS385-A-B_AC022486-RC | DYS385a/b |
| | _29490_29634 | |

In cases where conventional priming strategies are in conflict with parameters dictated by measurement of amplification products by mass spectrometry, alternative priming schemes were investigated. For example, the conventional products of the DYS385a/b locus are appreciably longer than the amplification products of other loci (241-324 nucleobases for the shortest primer set listed in the STRbase (Ruitberg, C. M.; Reeder, D. J.; Butler, J. M. Nucleic Acids Res. 2001, 29, 20-322; Wu, F. C.; Pu, C. E.; Forensic Sci. Int. 2001, 120, 213-222; Furedi, S., et al. Int. J. Legal Med. 1999, 113, 38-42; Schneider, P. M., et al. Forensic Sci. Int. 1998, 97, 61-70). There is substantial length contributed to the PCR product by an extended A/G region upstream of the ‘GAAA’ repeat. To take advantage of a distinct pattern of ‘A’ and ‘G’ present in this region, a primer binding site was chosen to reduce the product length range to 109-193 nucleobases. In another example, DYS389/II is one of the conventional loci of the 12 core Y-STR loci. In conventional Y-STR typing methods, the primer pair produces two amplification products, a smaller product designated DYS3 891 and a larger product designated DYS389II. This occurs because there is a duplicated binding site in the locus for the forward primer. In the present work, this complexity is eliminated by amplification of two regions separately and thus, primer pairs have been designed for each of two sub-loci, DYS389I and DYS389II. This is accomplished using a 3′ end difference in the forward primer binding region to favor formation of the shorter DYS389I product. The same forward primer with the first region at the 3′ end is used along with a reverse primer extending upstream of the second forward primer site to favor formation of the first part of DYS389II which is designated in the primer pair name as DYS389II-1 (excluding the repeat region of DYS389I). It was recognized that these two amplification products should not be produced in the same multiplex reaction.

A database was assembled which includes expected masses and base compositions of expected STR-identifying amplicons comprising the STR region and the flanking sequences to which the primers hybridize for each characterized allele. The base compositions and molecular masses were indexed to the primer pairs and alleles in the database.

Table 4 displays the reference alleles used to design primers for each of the 11 core Y-STR loci, along with the corresponding GenBank Accession number. Minimum and maximum product lengths were calculated using all characterized alleles. Each of the primers includes a 5′ T residue for the purpose of minimizing non-templated adenylation produced by Taq polymerase.

TABLE 4
Reference Alleles and Expected Amplicon Lengths for
Primer Pairs for Amplification of Core Y-STR Loci

| | | | Length of |
|---|---|---|---|
| | Reference | Reference GenBank | amplicons |
| Locus | Allele | Accession Number | (Range) |
| DYS19 | 15 | AC017019-RC | 159-207 |
| DYS385a/b | 11 | AC022486-RC | 109-290 |
| DYS389I | 12 | AC004617-RC | 88-180 |
| DYS389II | 29 | AC004617-RC | 106-146 |
| DYS390 | 24 | AC011289 | 140-201 |
| DYS391 | 11 | AC011289 | 82-161 |
| DYS392 | 13 | AC011745-RC | 87-138 |
| DYS393 | 12 | AC006152 | 100-145 |
| DYS437 | 16 | AC002992 | 120-187 |
| DYS438 | 10 | AC002531 | 94-177 |
| DYS439 | 13 | AC002992 | 113-143 |

Primer pairs designed to the 11 core Y-STR loci are listed in Table 5. The forward and reverse primer names in this table follow standard primer pair naming as described above.

TABLE 5
Primer Pairs Designed for Use in Human Y-STR DNA Analysis

| Primer | Forward | | Forward | | | Reverse |
|---|---|---|---|---|---|---|
| Pair | Primer | Forward | SEQ ID | Reverse | Reverse | SEQ ID |
| No. | Name | Sequence | NO | Primer Name | Sequence | NO |
| 4578 | DYS19_AC | TCACTATGA | 16 | DYS19_ | TCCATCTGG | 28 |
| | 017019- | CTACTGAGT | | AC017019- | GTTAAGGAG | |
| | RC_118941_ | TTCTGTTAT | | RC_119096_ | AGTGTC | |
| | 118971_F | AGTG | | 119119_R | | |
| 4579 | DYS19_AC | TGCCTACTG | 51 | DYS19_ | TCATCTGGG | 17 |
| | 017019- | AGTTTCTGT | | AC017019- | TTAAGGAGA | |
| | RC_118947_ | TATAGTGTT | | RC_119094_ | GTGTCAC | |
| | 118977_F | TTTT | | 119118_2_R | | |
| 4580 | DYS19_AC | TGACTACTG | 45 | DYS19_ | TGGGTTAAG | 60 |
| | 017019- | AGTTTCTGT | | AC017019- | GAGAGTGTC | |
| | RC_118947_ | TATAGTGTT | | RC_119088_ | ACTATATC | |
| | 118977_2_F | TTTT | | 119113_R | | |
| 4581 | DYS385-A- | TCAACAAAG | 10 | DYS385-A- | TCCAATTAC | 27 |
| | B_AC022486 | AAAAGAAAT | | B_AC022486- | ATAGTCCTC | |
| | -RC_29394_ | GAAATTCAG | | RC_29585_ | CTTTCTTTT | |
| | 29425_F | AAAGG | | 29615_R | TCTC | |
| 4582 | DYS385- | TGAAAGAGA | 42 | DYS385-A-B- | TCCAATTAC | 27 |
| | A-B-2_ | AAGAGGAAA | | 2_AC022486- | ATAGTCCTC | |
| | AC022486 | GAGAAAGAA | | RC_29585_ | CTTTCTTTT | |
| | -RC_29491_ | AGG | | 29615_R | TCTC | |
| | 29520_F | | | | | |
| 4583 | DYS385- | TCAACAAAG | 10 | DYS385-A-B- | TCCTTTCTT | 35 |
| | A-B- | AAAAGAAAT | | 1_AC022486- | TCTCTTTCC | |
| | 1_AC022486- | GAAATTCAG | | RC_29492_ | TCTTTCTCT | |
| | RC_29394_ | AAAGG | | 29521_R | TTC | |
| | 29425_F | | | | | |
| 4584 | DYS389I- | TCCAACTCT | 24 | DYS389I- | TGATAGATT | 46 |
| | II_AC004617- | CATCTGTAT | | II_AC004617- | GATAGAGGG | |
| | RC_125888_ | TATCTATGT | | RC_126077_ | AGGGATAGA | |
| | 125917_F | GTG | | 126106_R | TAG | |
| 4585 | DYS389I_ | TCCAACTCT | 23 | DYS389I_ | TCACAGTTA | 15 |
| | AC004617-RC_ | CATCTGTAT | | AC004617- | TCCCTGAGT | |
| | 126008_ | TATCTATGT | | RC_126138_ | AGTAGAAGA | |
| | 126039_F | ATCTG | | 126167_R | ATG | |
| 4586 | DYS389I_ | TCCAACTCT | 23 | DYS389I_ | TAGATAGAT | 5 |
| | AC004617-RC_ | CATCTGTAT | | AC004617- | TGATAGAGG | |
| | 126008_ | TATCTATGT | | RC_126077_ | GAGGGATAG | |
| | 126039_F | ATCTG | | 126107_R | ATAG | |
| 4587 | DYS389II- | TCCAACTCT | 24 | DYS389II- | TGATGAGAG | 47 |
| | 1_AC004617- | CATCTGTAT | | 1_AC004617- | TTGGATACA | |
| | RC_125888_ | TATCTATGT | | RC_125989_ | GAAGTAGGT | |
| | 125917_F | GTG | | 126021_R | ATAATG | |
| 4588 | DYS390_ | TGGGCCCTG | 59 | DYS390_ | TCATTGCAA | 20 |
| | AC011289_ | CATTTTGGT | | AC011289_ | TGTGTATAC | |
| | 11029_ | AC | | 11182_11210_R | TCAGAAACA | |
| | 11048_F | | | | AG | |
| 4589 | DYS390_ | TCATTTTTG | 21 | DYS390_ | TGCAATGTG | 49 |
| | AC011289_ | GGCCCTGCA | | AC011289_ | TATACTCAG | |
| | 11022_ | TTTTG | | 11177_11206_R | AAACAAGGA | |
| | 11044_F | | | | AAG | |
| 4590 | DYS390_ | TGGGCCCTG | 59 | DYS390_ | TGCAATGTG | 49 |
| | AC011289_ | CATTTTGGT | | AC011289_ | TATACTCAG | |
| | 11029_ | AC | | 11177_11206_R | AAACAAGGA | |
| | 11048_F | | | | AAG | |
| 4591 | DYS390_ | TCTGCATTT | 39 | DYS390_ | TGTGTATAC | 68 |
| | AC011289_ | TGGTACCCC | | AC011289_ | TCAGAAACA | |
| | 11034_ | ATAATATAT | | 11170_11201_R | AGGAAAGAT | |
| | 11062_F | TC | | | AGATA | |
| 4592 | DYS391_ | TCCCTTCAT | 32 | DYS391_ | TGCATAGCC | 50 |
| | G09613_18_ | TCAATCATA | | G09613_159_ | AAATATCTC | |
| | 44_F | CACCCATAT | | 181_R | CTGGG | |
| 4593 | DYS391_ | TCATTCAAT | 19 | DYS391_ | TCAATTGCC | 13 |
| | G09613_23_ | CATACACCC | | G09613_112_ | ATATCTGTC | |
| | 51_F | ATAGAGGGA | | 137_R | TAGGTAGG | |
| | | TG | | | | |
| 4594 | DYS391_ | TCATTCAAT | 19 | DYS391_ | TGCAAGCAA | 48 |
| | G09613_23_ | CATACACCC | | G09613_122_ | TTGCCATAG | |
| | 51_F | ATATCTGTC | | 142_R | AGG | |
| | | TG | | | | |
| 4595 | DYS391_ | TTCAATCAT | 70 | DYS391_ | TGGATAGGT | 57 |
| | G09613_26_ | ACACCCATA | | G09613_101_ | AGGCAGGCA | |
| | 53_F | TCTGTCTGT | | 123_2_R | GATAG | |
| | | C | | | | |
| 4596 | DYS392_ | TCCAAGCCA | 26 | DYS392_ | TCAACCTAC | 11 |
| | AC011745- | AGAAGGAAA | | AC011745-RC_ | CAATCCCAT | |
| | RC_97244_ | ACAAA | | 97336_97358_R | TCCTT | |
| | 97266_ | | | | | |
| 4597 | FDYS392_ | TGGAAAACA | 53 | DYS392_ | TCCATTAAA | 29 |
| | AC011745- | AATTTTTTC | | AC011745-RC_ | CCTACCAAT | |
| | RC_97256_ | CTTGTATCA | | 97338_97363_ | CCCATTCC | |
| | 97285_F | CCA | | 2_R | | |
| 4598 | DYS392_ | TCCAAGAAG | 25 | DYS392_ | TCATTAAAC | 18 |
| | AC011745- | GAAAACAAA | | AC011745-RC_ | CTACCAATC | |
| | RC_97249_ | TTTTTTCCT | | 97334_97362_R | CCATTCCTT | |
| | 97277_2_F | TG | | | AG | |
| 4599 | DYS392_ | TGTTATTTA | 69 | DYS392_ | TCATTAAAC | 18 |
| | AC011745- | AAAGCCAAG | | AC011745-RC_ | CTACCAATC | |
| | RC_97237_ | AAGGAAAAC | | 97334_97362_R | CCATTCCTT | |
| | 97266_F | AAA | | | AG | |
| 4600 | DYS393_ | TAATGTGGT | 1 | DYS393_ | TGAACTCAA | 43 |
| | AC006152_ | CTTCTACTT | | AC006152_ | GTCCAAAAA | |
| | 21087_ | GTGTCAATA | | 21182_21211_R | ATGAGGTAT | |
| | 21114_F | C | | | GTC | |
| 4601 | DYS393_ | TGGTGGTCT | 63 | DYS393_ | TGGAACTCA | 54 |
| | AC006152_ | TCTACTTGT | | AC006152_ | AGTCCAAAA | |
| | 21089_ | GTCAATAC | | 21188_21212_R | AATGAGG | |
| | 21114_F | | | | | |
| 4602 | DYS393_ | TGTGGTCTT | 67 | DYS393_ | TCAAGTCCA | 12 |
| | AC006152_ | CTACTTGTG | | AC006152_ | AAAAATGAG | |
| | 21090_ | TCAATACAG | | 21176_21206_R | GTATGTCTC | |
| | 21120_F | ATAG | | | ATAG | |
| 4603 | DYS393_ | TGGTCTTCT | 62 | DYS393_ | TGTCCAAAA | 64 |
| | AC006152_ | ACTTGTGTC | | AC006152_ | AATGAGGTA | |
| | 21092_ | AATACAGAT | | 21176_21203_R | TGTCTCATA | |
| | 21120_F | AG | | | G | |
| 4604 | DYS437_ | TGTGAGTGC | 65 | DYS437_ | TGACCCTGT | 44 |
| | AC002992_ | ATGCCCATC | | AC002992_ | CATTCACAG | |
| | 42957_ | C | | 43109_43139_R | ATGATATAG | |
| | 42975_F | | | | ATAG | |
| 4605 | DYS437_ | TCGTGAGTG | 36 | DYS437_ | TCACAGATG | 14 |
| | AC002992_ | CATGCCCAT | | AC002992_ | ATATAGATA | |
| | 42956_ | C | | 43094_43127_R | GATAGATAA | |
| | 42974_2_F | | | | CCACAGA | |
| 4606 | DYS437_ | TATGGGCGT | 8 | DYS437_ | TCACAGATG | 14 |
| | AC002992_ | GAGTGCATG | | AC002992_ | ATATAGATA | |
| | 42951_ | C | | 43094_43127_R | GATAGATAA | |
| | 42969_F | | | | CCACAGA | |
| 4607 | DYS437_ | TCTATGGGC | 38 | DYS437_ | TGGTAAATA | 61 |
| | AC002992_ | GTGAGTGCA | | AC002992_ | TCATTCATA | |
| | 42949_ | TG | | 43061_43096_ | GATAAGTAG | |
| | 42968_F | | | 2_R | ATAGACATC | |
| 4608 | DYS437_ | TCGTGAGTG | 36 | DYS437_ | TCGTTCATA | 37 |
| | AC002992_ | CATGCCCAT | | AC002992_ | GATAAGTAG | |
| | 42956_ | C | | 43055_ | ATAGACATC | |
| | 42974_2_F | | | 43087_R | ATTCAC | |
| 4609 | DYS438_ | TAGTGGGGA | 7 | DYS438_ | TGGAGGTTG | 56 |
| | AC002531_ | ATAGTTGAA | | AC002531_ | TGGTGAGTC | |
| | 129796_ | CGGTAA | | 129932_ | GAG | |
| | 129819_F | | | 129952_R | | |
| 4610 | DYS438_ | TTGGGGAAT | 71 | DYS438_ | TCTGGGCAA | 41 |
| | AC002531_ | AGTTGAACG | | AC002531_ | CAAGAGTGA | |
| | 129798_ | GTAAACAG | | 129889_ | AACTC | |
| | 129823_2_F | | | 129911_2_R | | |
| 4611 | DYS438_ | TCCAAAATT | 22 | DYS438_ | TAGCCTGGG | 6 |
| | AC002531_ | AGTGGGGAA | | AC002531_ | CAACAAGAG | |
| | 129788_ | TAGTTGAAC | | 129895_ | TG | |
| | 129815_F | G | | 129914_R | | |
| 4612 | DYS438_ | TTGGGGAAT | 71 | DYS438_ | TATTTCAGC | 9 |
| | AC002531_ | AGTTGAACG | | AC002531_ | CTGGGCAAC | |
| | 129798_ | GTAAACAG | | 129897_129919_ | AAGAG | |
| | 129823_2_F | | | 2_R | | |
| 4613 | DYS439_ | TAGATACAT | 3 | DYS439_ | TGGCCTGGC | 58 |
| | AC002992_ | AGGTGGAGA | | AC002992_ | TTGGAATTC | |
| | 91258_ | CAGATAGAT | | 91375_91396_R | TTTT | |
| | 91287_F | GAT | | | | |
| 4614 | DYS439_ | TACATAGGT | 2 | DYS439_ | TCTGGCTTG | 40 |
| | AC002992_ | GGAGACAGA | | AC002992_ | GAATTCTTT | |
| | 91262_ | TAGATGATA | | 91368_91393_R | TACCCATC | |
| | 91293_F | AATAG | | | | |
| 4615 | DYS439_ | TAGATAGAT | 4 | DYS439_ | TGCTTGGAA | 52 |
| | AC002992_ | ACATAGGTG | | AC002992_ | TTCTTTTAC | |
| | 91254_ | GAGACAGAT | | 91363_91390_R | CCATCATCT | |
| | 91285_F | AGATG | | | C | |
| 4616 | DYS439_ | TACATAGGT | 2 | DYS439_ | TGCTTGGAA | 52 |
| | AC002992_ | GGAGACAGA | | AC002992_ | TTCTTTTAC | |
| | 91262_ | TAGATGATA | | 91363_91390_R | CCATCATCT | |
| | 91293_F | AATAG | | | C | |
| 4670 | DYS393_ | TCCTCTTCT | 62 | DYS393_ | TGGAGGTAT | 55 |
| | AC006152_ | ACTTGTGTC | | AC006152_ | GTCTCATAG | |
| | 21092_ | AATACAGAT | | 21165_21193_R | AAAAGACAT | |
| | 21120_F | AG | | | AC | |
| 4671 | DYS393_ | TCCTCTTCT | 33 | DYS393_ | TCCCCAAAA | 31 |
| | AC006152_ | ACTTGTGTC | | AC006152_ | AATGAGGTA | |
| | 21092_ | AATACAGAT | | 21176_21203_ | TGTCTCATA | |
| | 21120_2_F | AG | | 2_R | G | |
| 4672 | DYS393_ | TCCTGGTCT | 34 | DYS393_ | TCCCACTCA | 30 |
| | AC006152_ | TCTACTTGT | | AC006152_ | AGTCCAAAA | |
| | 21089_ | GTCAATAC | | 21188_21212_ | AATGAGG | |
| | 21114_2_F | | | 2_R | | |
| 4673 | DYS390_ | TTTCCATTT | 73 | DYS390_ | TTTTGTATA | 74 |
| | AC011289_ | TGGTACCCC | | AC011289_ | CTCAGAAAC | |
| | 11034_ | ATAATATAT | | 11169_11202_R | AAGGAAAGA | |
| | 11066_F | TCTATC | | | TAGATAG | |
| 4691 | DYS385-A-B- | TGAAAGAGA | 42 | DYS385-A- | TGTGGGATA | 66 |
| | 2_AC022486- | AAGAGGAAA | | B_AC022486- | ATCTATCTA | |
| | RC_29491_ | GAGAAAGAA | | RC_29601_ | TTCCAATTA | |
| | 29520_F | AGG | | 29634_R | CATAGTC | |
| 4692 | DYS385-A- | TTTAAAGAG | 72 | DYS385-A- | TGTGGGATA | 67 |
| | B_AC022486- | AAAGAGGAA | | B_AC022486- | ATCTATCTA | |
| | RC_29490_ | AGAGAAAGA | | RC_29601_ | TTCCAATTA | |
| | 29520_F | AAGG | | 29634_R | CATAGTC | |

### Example 8

### Initial Primer Pair Testing Using PCR and Mass Spectrometry

Initial primer testing was carried out using standard PCR reactions similar to the methods described herein. Each 40 μl reaction contained 10 mM Tris-Cl, 75 mM KCl, 1.5 mM MgCl₂, 400 mM betaine, 200 μM each of dATP, dCTP, and dTTP (BioLine), 200 μM ¹³C-enriched dGTP (Cambridge Isotope Laboratories), and 1.5 U/reaction of Immolase™ DNA polymerase (BioLine). All primers were tested in duplicate in single primer pair reactions using 1 ng of template DNA (male blood sample SC35495 from SeraCare, Inc.). The thermocycling steps included 96° C. for 10 min, 40 cycles of (96° C., 25 sec, 56° C., 1.5 min, 72° C., 40 sec), followed by 72° C. for 4 min, and a 4° C. hold. Amplification products were analyzed by mass spectrometry as described herein.

The first test of the Y-STR primer pairs suggested that there was at least one primer pair per locus that was likely to perform to a sufficient extent to carry forward to a final assay. The results of this test produced three groups of primer pairs, one group to carry forward as assay candidate primers, one group of backup primers to be further tested or redesigned as backups and one group to be discarded due to poor performance. Reasons for discarding primer pairs or relegating primer pairs to the backup group included any or all of the following reasons: ineffective priming (poor signal representing an amplification product) high extent of adenylation, production of more than one product, production of a large product, and high baseline noise in mass spectra. Table 6 provides the results of this first round of testing of the original group of primer pairs.

TABLE 6
Results of Initial Testing of Primer Pairs

| Primer | | | | |
|---|---|---|---|---|
| Pair No. | Locus | Continue | Backup | Discard |
| 4578 | DYS19 | | | X |
| 4579 | DYS19 | X | | |
| 4580 | DYS19 | | X | |
| 4581 | DYS385a/b | | X | |
| 4582 | DYS385a/b | X | | |
| 4584 | DYS389II | | X | |
| 4585 | DYS389I | | X | |
| 4586 | DYS389I | X | | |
| 4587 | DYS389II-1 | X | | |
| 4588 | DYS390 | | X | |
| 4589 | DYS390 | | X | |
| 4590 | DYS390 | | X | |
| 4591 | DYS390 | X | | |
| 4592 | DYS391 | | X | |
| 4593 | DYS391 | | X | |
| 4594 | DYS391 | X | | |
| 4595 | DYS391 | | X | |
| 4596 | DYS392 | | X | |
| 4597 | DYS392 | X | | |
| 4598 | DYS392 | | X | |
| 4599 | DYS392 | | | X |
| 4600 | DYS393 | | X | |
| 4601 | DYS393 | | X | |
| 4602 | DYS393 | X | | |
| 4603 | DYS393 | | X | |
| 4604 | DYS437 | | X | |
| 4605 | DYS437 | | X | |
| 4606 | DYS437 | | X | |
| 4607 | DYS437 | | X | |
| 4608 | DYS437 | X | | |
| 4609 | DYS438 | | | X |
| 4610 | DYS438 | | X | |
| 4611 | DYS438 | X | | |
| 4612 | DYS438 | | | X |
| 4613 | DYS439 | | X | |
| 4614 | DYS439 | | X | |
| 4615 | DYS439 | X | | |
| 4616 | DYS439 | | X | |

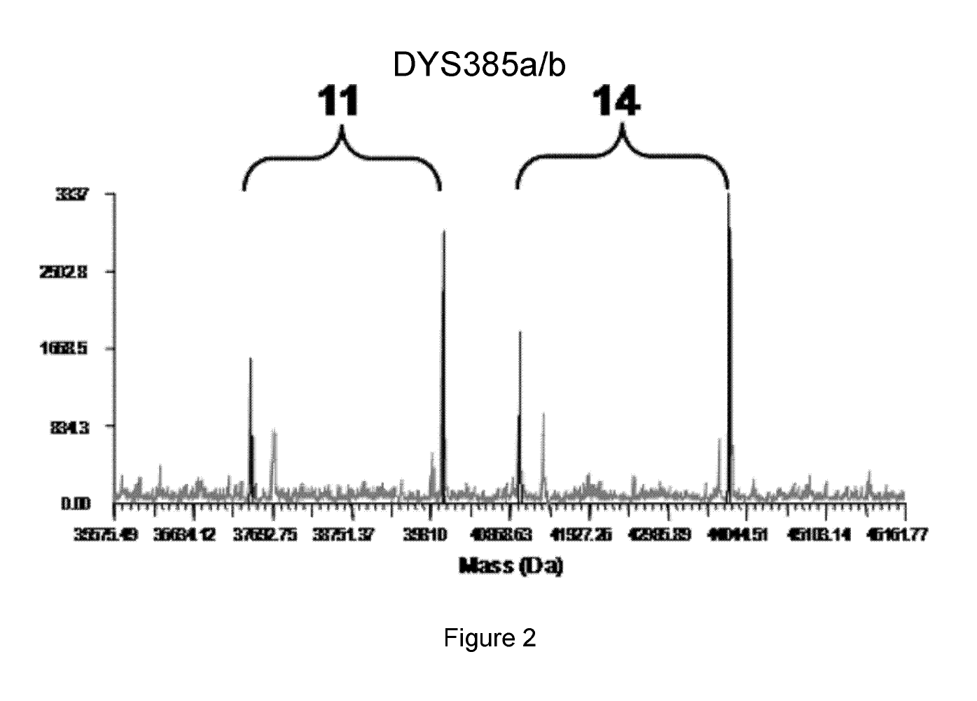

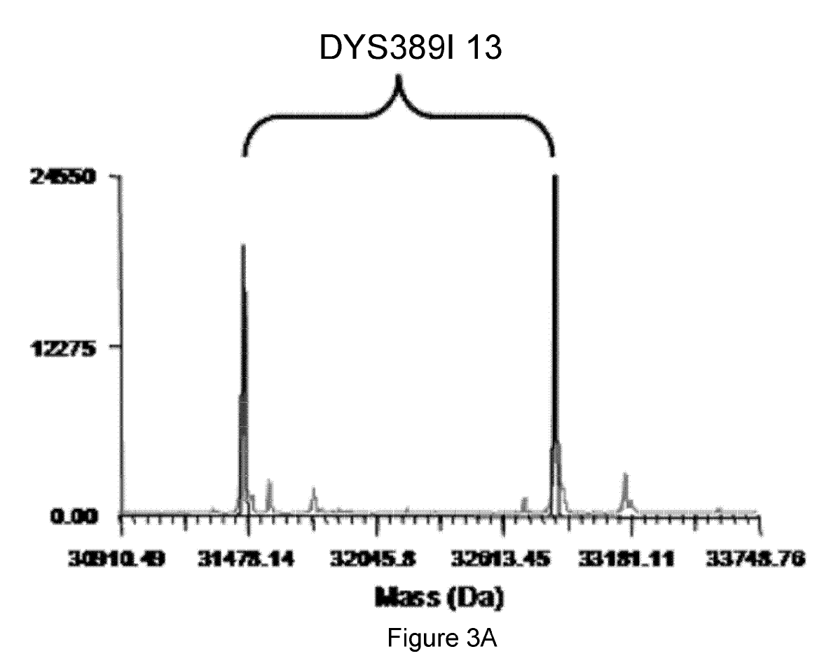

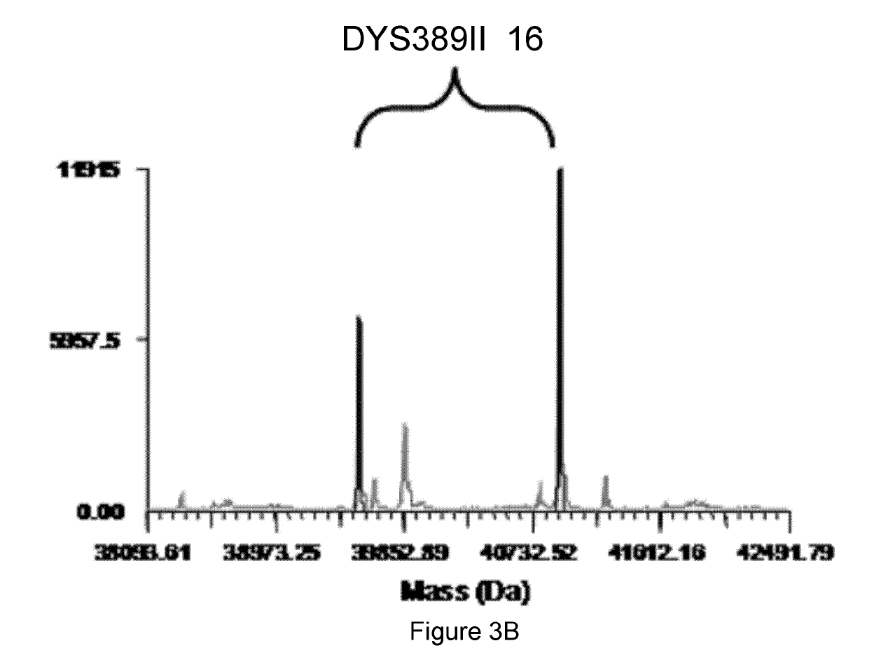

Importantly, the strategy used to shorten the products from DYS385a/b to a maximum size of less than 200 nucleobases and to split the DYS389I/II locus appeared to be working effectively. FIG. 2 indicates that primer pair number 4582 which was designed to exploit the non-repeating low complexity A/G-rich region near the repeat region of DYS385a/b successfully produces shorter amplification products which are clearly resolvable in the mass spectrum. Two amplification products are produced in this case because the DYS385 locus appears twice in the Y chromosome, hence the naming of the locus contains “a/b.” FIGS. 3A and 3B indicate that the two primer pairs used to split the DYS389I/II locus are successful in producing amplification products.

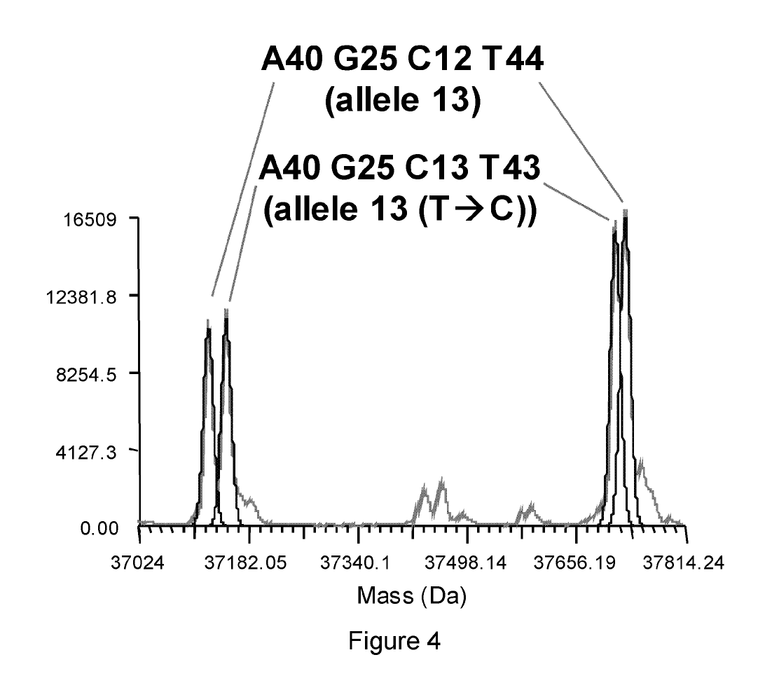

Interestingly, an additional allele was amplified with the primer pairs for DUS393 (see FIG. 4). There were four primer pairs designed to amplify DYS393. Two of these primer pairs (4602 and 4603) clearly produced an allele 13 and an additional product with a base composition consistent with an allele 13 with a T→C SNP. One primer pair (4600) produced an allele 13 and a product consistent with allele 13 with a C→G SNP. The other primer pair (4601) produced only one product (allele 13). The initial primer pair panel chosen was intended to exploit the additional discriminating information that may be revealed by the presence of an additional allele at DYS393. The hypothesis was that the locus may have been duplicated and that the individual used for testing had a SNP in one of the two loci. Conventional typing would not have detected this SNP. Testing of population samples (to be discussed below) has shown this hypothesis to be incorrect, as two alleles were produced in all samples and many of them are different lengths. The second allele contained a T→C SNP in every case, but appeared at lengths consistent with DYS393 alleles 12, 13, 14, 15 and 16. It subsequently appeared that the second allele is a homologous locus from the X-chromosome (Dupuy, B. M. et al. Forensic Sci. Int. 2000, 112, 111-21; Mayntz-Press, K. A.; Ballantyne, J. J. Forensic Sci. 2007, 52, 1025-34). As a result, it was concluded that the assay panel should be modified by switching to primer pair 4601 (see Table 5) or a derivative thereof which maintains the 3’ ends of 4601 in order to exclude the X-chromosome homolog.

### Example 9

### Development of Multiplexing Scheme for Y-STR Primer Pair Panel

Development of multiplexed reactions is a worthwhile endeavor because it enables more assays to be carried out within a single reaction vessel and therefore increases the efficiency of Y-STR typing processes. Multiplexing tests were initiated using the primer pairs and concentrations shown in Table 7. An aspect of multiplexing which must be considered is the possibility of overlapping signals due to DNA strands that have similar molecular masses. The primer pairs combined in multiplex reaction 1 and multiplex reaction 2 were thus chosen with respect to having sufficient separation in the sizes and masses of the amplification products that they would provide for the known alleles.

The same buffer and thermocycling conditions were used as described above for single-plex testing. Primer pairs in multiplexes were used at equal concentrations designed to total 800 nM for all primers combined (average of 200 nM per primer for the 4-plex reaction, or 160 nM per primer for the 5-plex). Blood sample SC35495 was tested in duplicate using 1 ng/reaction of DNA.

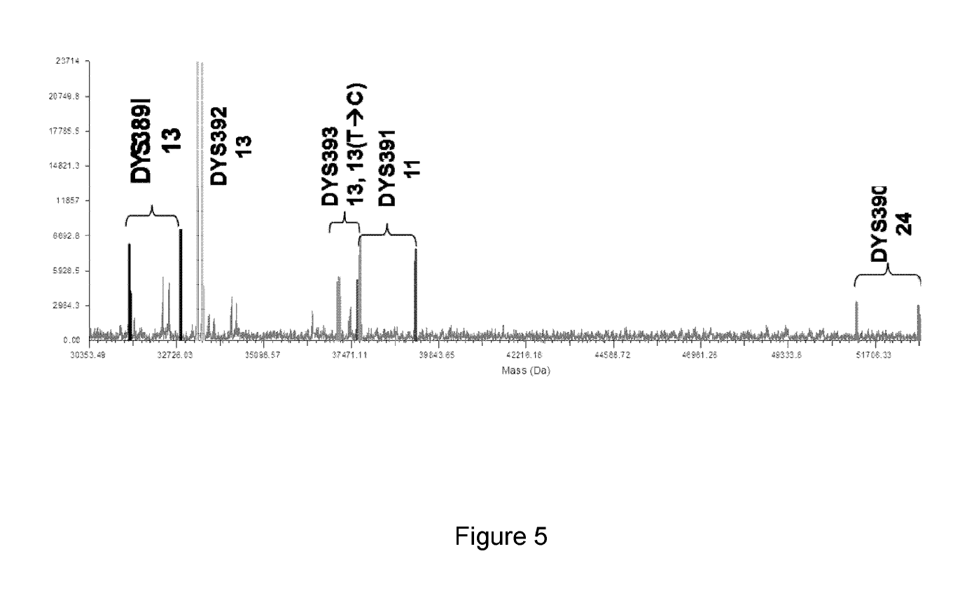

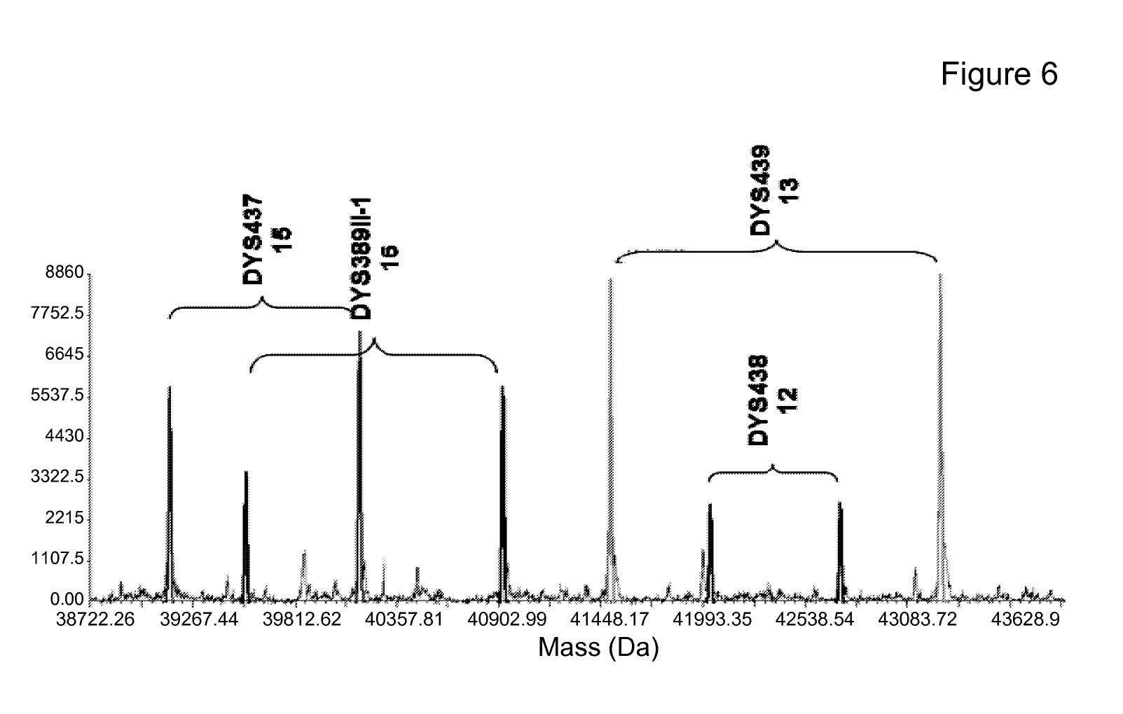

The mass spectrum of amplification products of the five-plex reaction containing primer pairs targeting DYS389I, DYS392, DYS391, DYS393 and DYS390 is shown in FIG. 5 and the mass spectrum of the amplification products of the four-plex reaction containing primer pairs targeting DYS389II-1, DYS438, DYS439 and DYS437 is shown in FIG. 6. As indicated, each strand of each amplification product can be resolved from the strands of the other amplification products and unambiguously assigned.

The initial test indicated that the relative yields of the amplification products were not well balanced. Iteratively over a series of four experiments, a final set of the concentrations of the primer pairs in the two multiplex reactions was obtained to achieve more balanced yields of amplification products. Additionally, the original primer pair chosen for DYS385a/b (4582) was modified (4692) to obtain an increased product yield and to reduce the extent of adenylation (not shown). The thermocycling parameters were also modified to include a 99° C., 10 min. step at the end to reduce post-PCR non-templated adenylation of PCR products prior to analysis. The final reaction layout (four reactions per sample) allows 24 samples to be run on a single 96-well plate.

TABLE 7
Primer Pairs and Concentrations Used for Initial Multiplex Testing

| | | | | Initial | | Final |
|---|---|---|---|---|---|---|
| | | | First | Test | Second | Test |
| | Primer | | Test | Relative | Test | Relative |
| | Pair | | Conc. | Product | Conc. | Product |
| Reaction | No. | Locus | (nM) | Yield | (nM) | Yield |
| Multiplex | 4586 | DYS389I | 160 | 26.6 | 130 | 23.0 |
| 1 | 4597 | DYS392 | 160 | 42.2 | 75 | 25.5 |
| | 4594 | DYS391 | 160 | 11.0 | 150 | 22.3 |
| | 4602 | DYS393 | 160 | 5.3 | 120 | 11.9 |
| | 4591 | DYS390 | 160 | 14.9 | 325 | 17.3 |
| Multiplex | 4587 | DYS389II-1 | 200 | 37.4 | 200 | 19.3 |
| 2 | 4611 | DYS438 | 200 | 11.4 | 345 | 29.0 |
| | 4615 | DYS439 | 200 | 31.4 | 115 | 26.7 |
| | 4608 | DYS437 | 200 | 19.8 | 140 | 24.9 |
| Single- | 4579 | DYS19 | 250 | — | 250 | — |
| plex 1 | | | | | | |
| Single- | 4582 | DYS385a/b | 250 | — | 250 | — |
| plex 2 | 4692 | DYS385a/b | (4582) | | (4692) | |

### Example 10

### Testing of the Y-STR Primer Pair Panel Against Individual Samples

Using the primer pair panel shown in Table 7 (with primer pair number 4692 in place of primer pair number 4582) 95 male population samples obtained from the National Institute of Standards and Technology (NIST) were tested using 1 ng/reaction of template. These samples included 31 Caucasians, 32 African Americans and 32 Hispanics.

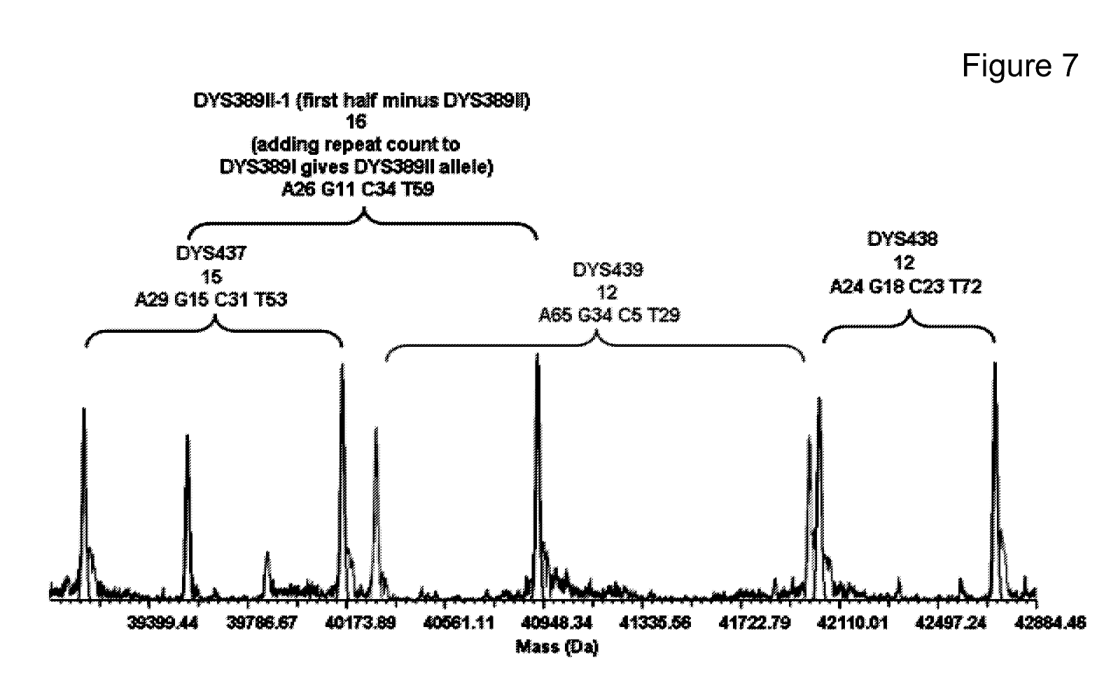

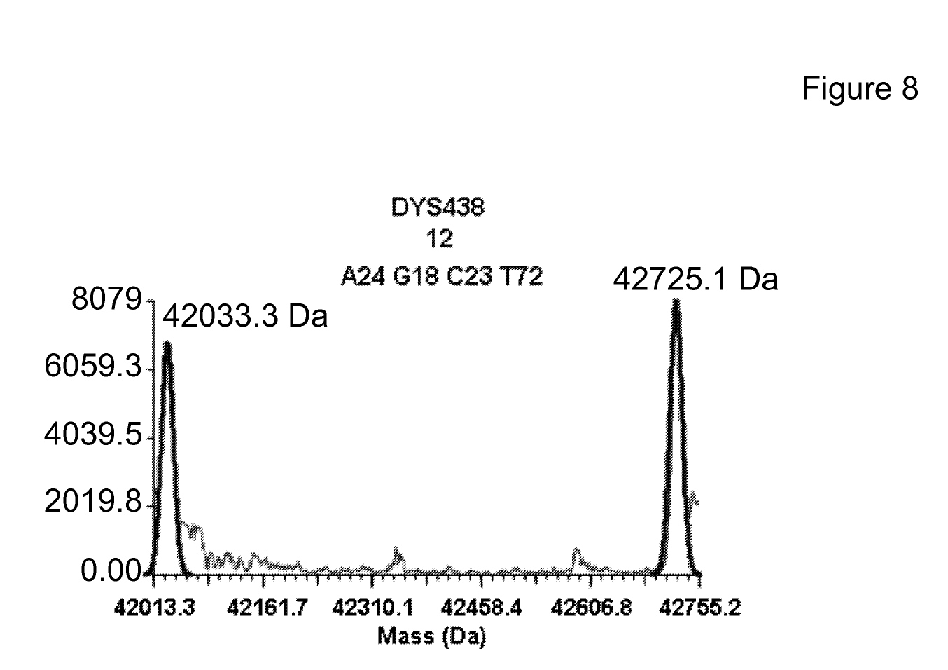

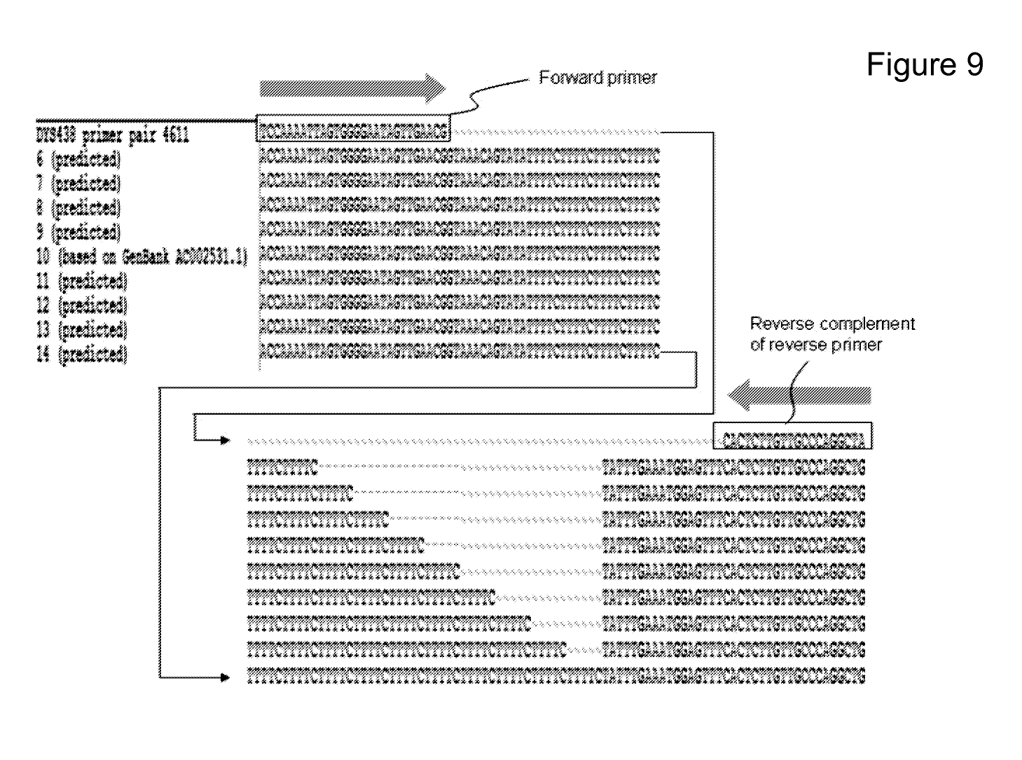

An example of a mass spectrum of an amplification product of the four-plex reaction of sample NIST-WT5137 is shown in FIG. 7 and an expanded view of the high mass end of the same spectrum is shown in FIG. 8 which indicates the amplification product obtained using primer pair number 4611 which targets the DYS438 locus. The base composition determined from the molecular mass of the amplification product is A24 G18 C23 T72. This matches the base composition of allele 12 as demonstrated in Table 8. The predicted sequences of the nine alleles are shown in a sequence alignment in FIG. 9 which also shows the hybridization coordinates or the forward and reverse primers for primer pair number 4611 with respect to the reference sequence AC002531.

TABLE 8
Lengths and Base Compositions of Amplification Products Obtained
with Primer Pair Number 4611 Targeting the DYS 438 Locus

| | Length of | |
|---|---|---|
| | Amplification | Base Composition of |
| Allele | Product | Amplification Product |
| 6 | 107 | A24 G18 C17 T48 |
| 7 | 112 | A24 G18 C18 T52 |
| 8 | 117 | A24 G18 C19 T56 |
| 9 | 122 | A24 G18 C20 T60 |
| 10 | 127 | A24 G18 C21 T64 |
| 11 | 132 | A24 G18 C22 T68 |
| 12 | 137 | A24 G18 C23 T72 |
| 13 | 142 | A24 G18 C24 T76 |
| 14 | 147 | A24 G18 C25 T80 |

Typing results are shown in Tables 9A and 9B. The additional column designated “deduced DYS389II” was derived by adding the allele numbers for DYS389I and DYS389II-1. The concordance of the allele for DYS389II-1 was deduced by the allele being equal to the truth data for DYS389II minus DYS389I.

All 95 samples produced full profiles with no apparent drop-outs. Base allele calls were consistent with truth data for the 92 samples for which truth data were available (truth data were not available for samples MT97172, UT57301 and WT51354, indicated by asterisks in Tables 9A and 9B). All 95 samples produced two alleles for locus DYS393. Unlike the control sample run for initial primer panel testing, however, the genotypes for DYS393 did not all consist of two same-length alleles. In fact, 78% of the samples had two different-length alleles at DYS39 as noted above in Example 8. Each sample had one allele at DYS393 that was consistent with a known allele with a T→C SNP and in every case the other allele was consistent with a non-polymorphic allele. For these 95 samples, the non-polymorphic allele was consistent with the truth data in all 92 cases where there was truth data. The initial interpretation of this result was that additional individual-differentiating information obtained with the second DYS393 allele could be exploited by inclusion of primer pair 4602 in our final primer panel. It appears, however, that the additional alleles are the result of amplifying the homolog of DYS393 from the X-chromosome (Dupuy, B. M. et al. Forensic Sci. Int. 2000, 112, 111-21; Mayntz-Press, K. A.; Ballantyne, J. J. Forensic Sci. 2007, 52, 1025-34).

In addition to being concordant with existing truth data, polymorphisms were revealed in four of the twelve loci listed in Tables 9A and 9B. The identification of these polymorphisms has resulted in the characterization of new alleles. Interestingly, the highest frequency of polymorphisms was seen in DYS389II. All of these were in the 5′ repeat region of the double locus (no polymorphisms were seen in DYS389I). For all 92 samples having truth data, the sum of the base allele numbers for DYS389I and DYS389II-1 was the same as the truth data allele number for DYS389I/II, suggesting that the strategy of splitting DYS389I/II into two separately analyzed products will still remain backwards-compatible with existing databases because the sum of the two alleles can be used to compare to existing genotypes for DYS389I/II.

TABLE 9A
Results of Y-STR Typing Results for African American Caucasian and Hispanic Populations

| | | | | | | Deduced | |
|---|---|---|---|---|---|---|---|
| Population | Sample | DYS19 | DYS385a/b | DYS389I | DYS389II-1 | DYS389II | DYS390 |
| African | JT51471 | 15 | 18, 18 | 13 | 18 (A→G) | 31 (A→G) | 21 |
| American | | | | | | | |
| African | JT51499 | 14 | 13, 14 | 12 | 16 | 28 | 22 |
| American | | | | | | | |
| African | OT05888 | 16 | 16, 17 | 14 | 17 (A→G) | 31 (A→G) | 22 |
| American | | | | | | | |
| African | OT05890 | 15 | 14, 15 | 12 | 17 (A→G) | 29 (A→G) | 22 |
| American | | | | | | | |
| African | OT05892 | 14 | 14, 14 | 12 | 16 | 28 | 23 |
| American | | | | | | | |
| African | OT05893 | 17 | 16, 18 | 13 | 17 (A→G) | 30 (A→G) | 21 |
| American | | | | | | | |
| African | OT05894 | 15 | 16, 17 | 14 | 17 (A→G) | 31 (A→G) | 21 |
| American | | | | | | | |
| African | OT05896 | 16 | 16, 18 | 13 | 18 (A→G) | 31 (A→G) | 21 |
| American | | | | | | | |
| African | OT05897 | 14 | 16 (G→A), | 13 | 17 (A→G) | 30 (A→G) | 21 |
| American | | | 17 | | | | |
| African | OT05898 | 15 | 12, 13 | 13 | 17 | 30 | 22 |
| American | | | | | | | (G→A) |
| African | OT05899 | 15 | 16, 17 | 13 | 18 (A→G) | 31 (A→G) | 22 |
| American | | | | | | | (A→G) |
| African | OT05901 | 14 | 11, 14 | 13 | 16 | 29 | 23 |
| American | | | | | | | |
| African | PT84214 | 17 | 17, 18 | 14 | 17 (A→G) | 31 (A→G) | 21 |
| American | | | | | | | |
| African | PT84215 | 15 | 16, 17 | 13 | 17 (A→G) | 30 (A→G) | 21 |
| American | | | | | | | |
| African | PT84216 | 13 | 16, 16 | 13 | 17 (A→G) | 30 (A→G) | 24 |
| American | | | | | | | |
| African | PT84222 | 14 | 13, 14 | 12 | 16 | 28 | 22 |
| American | | | | | | | |
| African | PT84223 | 15 | 15, 15 | 13 | 18 (A→G) | 31 (A→G) | 21 |
| American | | | | | | | |
| African | PT84224 | 15 | 16, 17 | 13 | 17 (A→G) | 30 (A→G) | 21 |
| American | | | | | | | |
| African | PT84225 | 15 | 13, 15 | 13 | 18 (A→G) | 31 (A→G) | 21 |
| American | | | | | | | |
| African | PT84226 | 15 | 16, 17 | 14 | 17 (A→G) | 31 (A→G) | 20 |
| American | | | | | | | |
| African | PT84227 | 15 | 16, 16 | 13 | 16 | 29 | 24 |
| American | | | | | | | |
| African | PT84228 | 14 | 12, 15 | 13 | 15 | 28 | 24 |
| American | | | | | | | |
| African | PT84230 | 16 | 16, 17 | 14 | 18 (A→G) | 32 (A→G) | 21 |
| American | | | | | | | |
| African | PT84231 | 16 | 18, 18 | 12 | 18 (A→G) | 30 (A→G) | 21 |
| American | | | | | | | |
| African | PT84232 | 15 | 11, 15 | 13 | 16 | 29 | 25 |
| American | | | | | | | |
| African | PT84234 | 15 | 14, 15 | 13 | 18 (A→G) | 31 (A→G) | 21 |
| American | | | | | | | |
| African | PT84236 | 14 | 11, 14 | 13 | 16 | 29 | 24 |
| American | | | | | | | |
| African | PT84239 | 15 | 16, 16 | 13 | 17 (A→G) | 30 (A→G) | 21 |
| American | | | | | | | |
| African | PT84240 | 16 | 11, 12 | 13 | 18 (A→G) | 31 (A→G) | 24 |
| American | | | | | | | |
| African | PT84241 | 16 | 11, 13 | 13 | 17 | 30 | 25 |
| American | | | | | | | |
| African | PT84242 | 15 | 15, 18 | 13 | 18 (A→G) | 31 (A→G) | 21 |
| American | | | | | | | |
| African | PT84243 | 16 | 16, 16 | 12 | 18 (A→G) | 30 (A→G) | 21 |
| American | | | | | | | |
| Caucasian | BC11352 | 15 | 15, 17 | 14 | 16 | 30 | 22 |
| Caucasian | MT94859 | 15 | 15, 15 | 14 | 17 | 31 | 23 |
| Caucasian | MT94866 | 14 | 11, 16 | 13 | 17 | 30 | 24 |
| Caucasian | MT94868 | 14 | 11, 14 | 13 | 16 | 29 | 23 |
| Caucasian | MT94869 | 14 | 11, 14 | 13 | 17 | 30 | 24 |
| Caucasian | MT94875 | 14 | 11, 14 | 13 | 16 | 29 | 24 |
| Caucasian | MT97172* | 16 | 13, 18 | 12 | 16 | 28 | 24 |
| Caucasian | UT57300 | 15 | 14, 14 | 13 | 17 | 30 | 23 |
| Caucasian | UT57301* | 14 | 12, 14 | 13 | 15 | 28 | 24 |
| Caucasian | UT57302 | 14 | 11, 15 | 13 | 16 | 29 | 24 |
| Caucasian | UT57303 | 15 | 14, 17 | 12 | 16 | 28 | 24 |
| Caucasian | UT57310 | 15 | 11, 14 | 12 | 19 | 31 | 25 |
| | | | | | | | (A→C) |
| Caucasian | UT57312 | 14 | 11, 15 | 13 | 16 | 29 | 24 |
| Caucasian | UT57317 | 16 | 13, 15 | 13 | 17 | 30 | 23 |
| Caucasian | UT57318 | 14 | 11, 14 | 14 | 16 | 30 | 24 |
| Caucasian | WA29584 | 14 | 11, 15 | 13 | 16 | 29 | 24 |
| Caucasian | WA29594 | 13 | 17, 18 | 14 | 17 (A→G) | 31 (A→G) | 25 |
| Caucasian | WA29612 | 14 | 12, 14 | 13 | 16 | 29 | 23 |
| Caucasian | WT51342 | 14 | 11, 14 | 14 | 16 | 30 | 24 |
| Caucasian | WT51343 | 14 | 11, 15 | 14 | 17 | 31 | 23 |
| Caucasian | WT51345 | 14 | 11, 14 | 13 | 17 | 30 | 23 |
| Caucasian | WT51354* | 14 | 12, 14 | 14 | 17 | 31 | 24 |
| Caucasian | WT51355 | 15 | 11, 14 | 14 | 16 | 30 | 24 |
| Caucasian | WT51358 | 16 | 11, 14 | 13 | 16 | 29 | 23 |
| Caucasian | WT51359 | 14 | 11, 14 | 13 | 16 | 29 | 24 |
| Caucasian | WT51362 | 14 | 11, 14 | 13 | 16 | 29 | 23 |
| Caucasian | WT51373 | 14 | 11, 14 | 14 | 16 | 30 | 25 |
| Caucasian | WT51378 | 14 | 11, 14 | 13 | 16 | 29 | 23 |
| Caucasian | WT51381 | 14 | 11, 14 | 13 | 16 | 29 | 24 |
| Caucasian | WT51386 | 15 | 13, 17 | 12 | 16 | 28 | 24 |
| Caucasian | ZT81387 | 15 | 11, 14 | 13 | 17 (G→A) | 30 (A→G) | 25 |
| | | | | | | | (A→C) |
| Hispanic | GT37778 | 16 | 16, 17 | 13 | 17 (A→G) | 30 (A→G) | 21 |
| Hispanic | GT37812 | 15 | 11, 14 | 14 | 16 | 30 | 23 |
| Hispanic | GT37828 | 15 | 15, 16 | 13 | 17 (G→A) | 30 (G→A) | 23 |
| | | | (G→A + | | | | |
| | | | G→C) | | | | |
| Hispanic | GT37862 | 13 | 14, 18 | 13 | 17 | 30 | 25 |
| Hispanic | GT37864 | 14 | 12, 16 | 13 | 16 | 29 | 24 |
| Hispanic | GT37869 | 14 | 11, 15 | 13 | 16 | 29 | 24 |
| Hispanic | GT37888 | 13 | 13, 14 | 14 | 16 (A→G) | 30 (A→G) | 24 |
| Hispanic | GT37900 | 16 | 13, 16 | 13 | 16 | 29 | 23 |
| Hispanic | GT37913 | 15 | 16, 18 | 12 | 16 | 28 | 24 |
| Hispanic | JT52076 | 14 | 13, 14 | 12 | 16 | 28 | 22 |
| Hispanic | OT07280 | 14 | 11, 14 | 12 | 17 | 29 | 24 |
| Hispanic | PT85612 | 15 | 16, 16 | 13 | 18 (A→G) | 31 (A→G) | 21 |
| Hispanic | PT85658 | 14 | 12 (A→G), | 13 | 16 | 29 | 24 |
| | | | 14 | | | | |
| Hispanic | TT51399 | 13 | 14, 17 | 13 | 17 (G→A) | 30 (A→G) | 24 |
| Hispanic | TT51407 | 15 | 16, 19 | 13 | 18 (A→G + | 31 (A→G) | 23 |
| | | | | | C→G) | | |
| Hispanic | TT51422 | 16 | 11, 13 | 12 | 17 | 29 | 25 |
| Hispanic | TT51435 | 15 | 13, 15 | 12 | 18 | 30 | 21 |
| Hispanic | TT51483 | 16 | 12, 12 | 13 | 15 | 28 | 23 |
| Hispanic | TT51511 | 15 | 11, 14 | 13 | 16 | 29 | 23 |
| Hispanic | TT51530 | 13 | 15, 18 | 12 | 16 | 28 | 23 |
| Hispanic | ZT80731 | 16 | 16, 16 | 14 | 17 (A→G) | 31 (A→G) | 21 |
| | | | | | | | (C→A) |
| Hispanic | ZT80737 | 14 | 11, 13 | 13 | 16 | 29 | 25 |
| Hispanic | ZT80782 | 14 | 10, 14 | 14 | 16 | 30 | 24 |
| Hispanic | ZT80786 | 14 | 13, 18 | 12 | 18 | 30 | 23 |
| Hispanic | ZT80815 | 14 | 13, 15 | 12 | 16 | 28 | 23 |
| | | | | | | | (A→G) |
| Hispanic | ZT80826 | 14 | 11, 14 | 14 | 16 | 30 | 23 |
| Hispanic | ZT80863 | 17 | 12, 12 | 13 | 15 | 28 | 23 |
| Hispanic | ZT80865 | 15 | 11, 14 | 13 | 17 | 30 | 24 |
| Hispanic | ZT80869 | 13 | 13, 15 | 12 | 16 | 28 | 24 |
| | | | | | | | (A→G) |
| Hispanic | ZT80870 | 15 | 13, 16 | 12 | 17 | 29 | 24 |
| Hispanic | ZT80925 | 13 | 15, 18 | 13 | 17 (A→G) | 30 (A→G) | 24 |
| Hispanic | ZT80932 | 14 | 11, 14 | 13 | 16 | 29 | 24 |

TABLE 9B
Results of Y-STR Typing Results for African American, Caucasian and Hispanic Populations

| Population | Sample | DYS391 | DYS392 | DYS393 | DYS437 | DYS438 | DYS439 |
|---|---|---|---|---|---|---|---|
| African | JT51471 | 10 | 11 | 13, 14 | 14 | 11 | 12 |
| American | | | | (T→C) | (C→T) | | |
| African | JT51499 | 11 | 11 | 13, 14 | 16 | 10 | 11 |
| American | | | | (T→C) | | | |
| African | OT05888 | 10 | 11 | 13, 14 | 14 | 11 | 11 |
| American | | | | (T→C) | (C→T) | | |
| African | OT05890 | 10 | 11 | 13, 15 | 17 | 8 | 12 |
| American | | | | (T→C) | (A→G) | | |
| African | OT05892 | 10 | 11 | 12, 14 | 16 | 10 | 11 |
| American | | | | (T→C) | | | |
| African | OT05893 | 10 | 11 | 15, 15 | 14 | 11 | 11 |
| American | | | | (T→C) | (C→T) | | |
| African | OT05894 | 10 | 11 | 14, 14 | 14 | 11 | 11 |
| American | | | | (T→C) | (C→T) | | |
| African | OT05896 | 10 | 11 | 13 | 14 | 11 | 11 |
| American | | | | (T→C), | (C→T) | | |
| | | | | 15 | | | |
| African | OT05897 | 10 | 11 | 13, 15 | 13 | 11 | 12 |
| American | | | | (T→C) | (C→T) | | |
| African | OT05898 | 10 | 12 | 13, 14 | 16 | 10 | 12 |
| American | | | | (T→C) | | | |
| African | OT05899 | 10 | 11 | 13, 14 | 14 | 11 | 12 |
| American | | | | (T→C) | (C→T) | | |
| African | OT05901 | 11 | 13 | 13, 14 | 15 | 12 | 11 |
| American | | | | (T→C) | | | |
| African | PT84214 | 10 | 11 | 13 | 14 | 11 | 12 |
| American | | | | (T→C), | (C→T) | | |
| | | | | 15 | | | |
| African | PT84215 | 10 | 11 | 13, 13 | 14 | 11 | 11 |
| American | | | | (T→C) | (C→T) | | |
| African | PT84216 | 10 | 13 | 13, 14 | 15 | 10 | 12 |
| American | | | | (T→C) | | | |
| African | PT84222 | 10 | 11 | 13, 14 | 16 | 10 | 11 |
| American | | | | (T→C) | | | |
| African | PT84223 | 10 | 12 | 13, 14 | 14 | 11 | 12 |
| American | | | | (T→C) | (C→T) | | |
| African | PT84224 | 11 | 11 | 13, 15 | 14 | 11 | 12 |
| American | | | | (T→C) | (C→T) | | |
| African | PT84225 | 10 | 11 | 13, 15 | 14 | 11 | 11 |
| American | | | | (T→C) | (C→T) | | |
| African | PT84226 | 10 | 11 | 13, 14 | 14 | 11 | 13 |
| American | | | | (T→C) | (C→T) | | |
| African | PT84227 | 10 | 12 | 15, 15 | 15 | 10 | 12 |
| American | | | | (T→C) | | | |
| African | PT84228 | 11 | 13 | 13, 15 | 15 | 12 | 11 |
| American | | | | (T→C) | | | |
| African | PT84230 | 11 | 11 | 13, 13 | 14 | 11 | 12 |
| American | | | | (T→C) | (C→T) | | |
| African | PT84231 | 10 | 11 | 13 | 14 | 11 | 12 |
| American | | | | (T→C), | (C→T) | | |
| | | | | 15 | | | |
| African | PT84232 | 10 | 13 | 12, 14 | 15 | 12 | 13 |
| American | | | | (T→C) | | | |
| African | PT84234 | 10 | 11 | 13, 17 | 14 | 11 | 11 |
| American | | | | (T→C) | (C→T) | | |
| African | PT84236 | 11 | 13 | 13, 13 | 14 | 12 | 12 |
| American | | | | (T→C) | | | |
| African | PT84239 | 10 | 11 | 13, 14 | 14 | 11 | 12 |
| American | | | | (T→C) | (C→T) | | |
| African | PT84240 | 10 | 7 | 13, 14 | 14 | 10 | 12 |
| American | | | | (T→C) | | | |
| African | PT84241 | 10 | 11 | 13, 14 | 14 | 11 | 12 |
| American | | | | (T→C) | | | |
| African | PT84242 | 10 | 11 | 14, 14 | 14 | 11 | 11 |
| American | | | | (T→C) | (C→T) | | |
| African | PT84243 | 10 | 11 | 13 | 14 | 11 | 12 |
| American | | | | (T→C), | (C→T) | | |
| | | | | 14 | | | |
| Caucasian | BC11352 | 10 | 11 | 12, 14 | 14 | 9 | 11 |
| | | | | (T→C) | | | |
| Caucasian | MT94859 | 10 | 12 | 12 | 14 | 10 | 11 |
| | | | | (T→C), | | | |
| | | | | 14 | | | |
| Caucasian | MT94866 | 11 | 15 | 12, 15 | 15 | 12 | 12 |
| | | | | (T→C) | | | |
| Caucasian | MT94868 | 11 | 13 | 13, 14 | 16 | 12 | 12 |
| | | | | (T→C) | | | |
| Caucasian | MT94869 | 10 | 13 | 13, 15 | 15 | 12 | 12 |
| | | | | (T→C) | | | |
| Caucasian | MT94875 | 11 | 13 | 13, 14 | 15 | 12 | 11 |
| | | | | (T→C) | | | |
| Caucasian | MT97172* | 10 | 11 | 12, 12 | 16 | 9 | 11 |
| | | | | (T→C) | | | |
| Caucasian | UT57300 | 10 | 12 | 13 | 14 | 10 | 11 |
| | | | | (T→C), | | | |
| | | | | 14 | | | |
| Caucasian | UT57301* | 11 | 13 | 13, 14 | 15 | 12 | 12 |
| | | | | (T→C) | | | |
| Caucasian | UT57302 | 11 | 13 | 13, 15 | 15 | 12 | 12 |
| | | | | (T→C) | | | |
| Caucasian | UT57303 | 10 | 11 | 12, 15 | 16 | 9 | 12 |
| | | | | (T→C) | | | |
| Caucasian | UT57310 | 10 | 11 | 13, 15 | 14 | 11 | 10 |
| | | | | (T→C) | | | |
| Caucasian | UT57312 | 11 | 13 | 13, 13 | 15 | 12 | 13 |
| | | | | (T→C) | | | |
| Caucasian | UT57317 | 9 | 11 | 12, 15 | 14 | 9 | 12 |
| | | | | (T→C) | | | |
| Caucasian | UT57318 | 11 | 13 | 13, 13 | 15 | 12 | 12 |
| | | | | (T→C) | | | |
| Caucasian | WA29584 | 11 | 13 | 13, 13 | 15 | 12 | 12 |
| | | | | (T→C) | | | |
| Caucasian | WA29594 | 9 | 11 | 13, 14 | 14 | 10 | 13 |
| | | | | (T→C) | | | |
| Caucasian | WA29612 | 11 | 13 | 12, 13 | 15 | 12 | 12 |
| | | | | (T→C) | | | |
| Caucasian | WT51342 | 11 | 13 | 13, 15 | 15 | 12 | 12 |
| | | | | (T→C) | | | |
| Caucasian | WT51343 | 11 | 13 | 13, 13 | 14 | 12 | 12 |
| | | | | (T→C) | | | |
| Caucasian | WT51345 | 12 | 13 | 13, 15 | 15 | 12 | 12 |
| | | | | (T→C) | | | |
| Caucasian | WT51354* | 9 | 13 | 13, 14 | 15 | 12 | 12 |
| | | | | (T→C) | | | |
| Caucasian | WT51355 | 11 | 13 | 13, 15 | 15 | 12 | 13 |
| | | | | (T→C) | | | |
| Caucasian | WT51358 | 10 | 13 | 13, 14 | 16 | 11 | 12 |
| | | | | (T→C) | | | |
| Caucasian | WT51359 | 11 | 13 | 13, 15 | 15 | 12 | 12 |
| | | | | (T→C) | | | |
| Caucasian | WT51362 | 11 | 13 | 13, 13 | 15 | 12 | 12 |
| | | | | (T→C) | | | |
| Caucasian | WT51373 | 11 | 13 | 12 | 15 | 12 | 12 |
| | | | | (T→C), | | | |
| | | | | 13 | | | |
| Caucasian | WT51378 | 11 | 13 | 12, 14 | 15 | 12 | 12 |
| | | | | (T→C) | | | |
| Caucasian | WT51381 | 11 | 11 | 13, 13 | 14 | 12 | 13 |
| | | | | (T→C) | | | |
| Caucasian | WT51386 | 10 | 11 | 12, 15 | 16 | 9 | 12 |
| | | | | (T→C) | | | |
| Caucasian | ZT81387 | 10 | 11 | 13, 13 | 14 | 11 | 10 |
| | | | | (T→C) | | | |
| Hispanic | GT37778 | 10 | 11 | 13, 13 | 14 | 11 | 12 |
| | | | | (T→C) | (C→T) | | |
| Hispanic | GT37812 | 11 | 14 | 12 | 15 | 12 | 11 |
| | | | | (T→C), | | | |
| | | | | 13 | | | |
| Hispanic | GT37828 | 11 | 13 | 13, 15 | 13 | 9 | 11 |
| | | | | (T→C) | | | |
| Hispanic | GT37862 | 10 | 16 | 13, 15 | 14 | 11 | 11 |
| | | | | (T→C) | | | |
| Hispanic | GT37864 | 11 | 13 | 13, 13 | 15 | 12 | 13 |
| | | | | (T→C) | | | |
| Hispanic | GT37869 | 11 | 14 | 13, 14.3 | 15 | 10 | 13 |
| | | | | (T→C) | | | |
| Hispanic | GT37888 | 9 | 11 | 13, 14 | 14 | 10 | 10 |
| | | | | (T→C) | | | |
| Hispanic | GT37900 | 9 | 11 | 12, 13 | 14 | 9 | 13 |
| | | | | (T→C) | | | |
| Hispanic | GT37913 | 10 | 11 | 12, 13 | 14 | 9 | 13 |
| | | | | (T→C) | | | |
| Hispanic | JT52076 | 10 | 11 | 13, 16 | 17 | 10 | 11 |
| | | | | (T→C) | (A→G) | | |
| Hispanic | OT07280 | 11 | 11 | 13, 13 | 15 | 12 | 13 |
| | | | | (T→C) | | | |
| Hispanic | PT85612 | 11 | 11 | 13, 15 | 14 | 11 | 11 |
| | | | | (T→C) | (C→T) | | |
| Hispanic | PT85658 | 11 | 13 | 13, 14 | 15 | 12 | 12 |
| | | | | (T→C) | | | |
| Hispanic | TT51399 | 10 | 16 | 13, 15 | 14 | 11 | 12 |
| | | | | (T→C) | | | |
| Hispanic | TT51407 | 10 | 11 | 13 | 14 | 11 | 13 |
| | | | | (T→C), | | | |
| | | | | 14 | | | |
| Hispanic | TT51422 | 10 | 11 | 13, 15 | 14 | 11 | 10 |
| | | | | (T→C) | | | |
| Hispanic | TT51435 | 10 | 11 | 13 | 16 | 10 | 12 |
| | | | | (T→C), | | | |
| | | | | 15 | | | |
| Hispanic | TT51483 | 10 | 11 | 13, 15 | 14 | 10 | 13 |
| | | | | (T→C) | | | |
| Hispanic | TT51511 | 11 | 13 | 13, 14 | 15 | 12 | 13 |
| | | | | (T→C) | | | |
| Hispanic | TT51530 | 10 | 13 | 14, 14 | 14 | 11 | 13 |
| | | | | (T→C) | | | |
| Hispanic | ZT80731 | 10 | 11 | 13, 15 | 14 | 11 | 13 |
| | | | | (T→C) | (C→T) | | |
| Hispanic | ZT80737 | 11 | 13 | 13, 15 | 15 | 12 | 12 |
| | | | | (T→C) | | | |
| Hispanic | ZT80782 | 11 | 13 | 13, 14 | 15 | 13 | 11 |
| | | | | (T→C) | | | |
| Hispanic | ZT80786 | 11 | 11 | 12, 12 | 14 | 10 | 11 |
| | | | | (T→C) | | (A→C) | |
| Hispanic | ZT80815 | 11 | 11 | 13, 14 | 16 | 10 | 11 |
| | | | | (T→C) | | | |
| Hispanic | ZT80826 | 10 | 13 | 12 | 14 | 12 | 12 |
| | | | | (T→C), | | | |
| | | | | 13 | | | |
| Hispanic | ZT80863 | 10 | 11 | 13, 15 | 15 | 10 | 12 |
| | | | | (T→C) | | | |
| Hispanic | ZT80865 | 11 | 13 | 13, 15 | 15 | 12 | 12 |
| | | | | (T→C) | | | |
| Hispanic | ZT80869 | 10 | 11 | 13, 13 | 16 | 10 | 11 |
| | | | | (T→C) | | | |
| Hispanic | ZT80870 | 10 | 11 | 12, 14 | 16 | 9 | 12 |
| | | | | (T→C) | | | |
| Hispanic | ZT80925 | 10 | 11 | 13, 14 | 14 | 10 | 12 |
| | | | | (T→C) | | | |
| Hispanic | ZT80932 | 11 | 13 | 12, 13 | 15 | 12 | 12 |
| | | | | (T→C) | | | |

### Example 11

### Specificity Testing Against Female DNA/X Chromosome

To test the preliminary primer pairs from Table 5 for specificity to the Y-chromosome, all 39 candidate primer pairs were tested individually with 1 ng/reaction of female DNA sample N31774 (SeraCare, Inc.). All reactions were done in duplicate. Primer pairs 4600, 4602 and 4603 all produced two clear products (not shown), showing alleles from both X-chromosomes. The genotype was consistent with DYS393 (de Knijff et al. Int. J. Legal Med. 1997, 110, 141-149; Dupuy, B. M. et al. T. Forensic Sci. Int. 2000. 112, 111-21). Primer pair number 4601 did not produce an appreciable product, and the signal output from both replicates corresponded to unconsumed primer pairs (not shown). For this reason, future work will include switching primer pair 4601 in for primer pair 4602 in the panel shown in Table 7.

In addition to DYS393, one primer pair for locus DYS389I (primer pair 4586) produced a single product from the female DNA that was smaller than the smallest DYS389I allele in the database (allele 9 which has a base composition of A18 G5 C26 T39). The product appeared to have a base composition of [A19 G4 C26 T35]. This composition is not consistent with a simple difference in TCTA and/or TCTG repeats. The alternative primer pair for DYS389I (4585) did not produce a product with female DNA (not shown). However, the products produced for primer pair 4585 are considerably larger than for 4586. Testing of male DNA in the presence of excess female DNA will be required to characterize the extent to which the possibility of cross-reactivity of primer pair 4585 will be a problem. In addition, tests with excess female DNA (beyond 10 ng/reaction) should be performed to understand if homologous loci on the X-chromosome will interfere with correct typing results for all finalized primer pairs (Mayntz-Press, K. A.; Ballantyne, J. J. Forensic Sci. 2007, 52, 1025-34).

### Example 12

### Y-STR Assay Process Control

The system used in measuring the molecular masses of the amplification products described herein includes a mass spectrometer in conjunction with a controller which is operably connected to the mass spectrometer. After the mass spectral data is acquired, the controller queries the database for primer pairs in each well and triggers an assessment of allelic mass ranges for each well. Data processing is automatically performed over a suitable mass range for each well in an assay plate. No manual interface is required for processing of amplification products.

The controller includes an integrated function to register and store STR and Y-STR profiles directly from the analysis interface. An additional interface is provided to query STR and Y-STR profiles that have been stored in the database by sample name, database and/or population. Profiles may be queried with polymorphisms or by base allele call only (for concordance comparisons or for backwards-compatibility). There is also a query option to show SNPs descriptively (e.g. A→G), or using a string that allows allele designations to be more easily analyzed in existing software packages.

The analysis interface is generalized to allow analysis of STRs, Y-STRs or autosomal SNPs or any other products that can be represented as labeled alleles. A sample status query has been added to allow tracking of the time points when profiles were run, the identifier of the source plate and the well in which each sample originates, as well as the identifier of the mass spectrometry plate(s).

A database-integrated repeat queue is implemented to improve the sample tracking efficiency. The controller includes a base composition browser enhanced for STR and Y-STR analyses (or analysis based upon named alleles) to allow browsing hypotheses by allele name as well as by base composition.

Signal processing functions are integrated to automatically assist in proper assignment of overlapping masses, such as the case of same-length heterozygous states where the alleles differ by an A⇄T SNP (in this case, masses would overlap because they differ by only 9 Da).

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

1. A method for identifying a known STR allele or characterizing a previously unknown STR allele in a nucleic acid sample, said method comprising:
a) selecting a nucleic acid locus comprising said STR allele;
b) amplifying at least a portion of said locus using an oligonucleotide primer pair comprising a forward and a reverse primer, each between 13 and 40 nucleobases in length, thereby generating an amplification product with a length of about 45 to about 200 nucleobases, wherein said amplification product duplicates the sequence of said known or unknown STR
c) measuring the molecular mass of one or both strands of said amplification product;
d) determining the base composition of said one or both strands;
e) comparing said base composition to a plurality of database-stored base compositions of strands of amplification products of known alleles of said locus; and
f) identifying a match between said base composition and at least one of said database-stored base compositions of amplification products comprising said sequence of said STR allele produced with said primer pair, thereby identifying said allele or, alternatively, failing to identity a match between said base composition and at least one of said database-stored base compositions, thereby characterizing a previously unknown STR allele.

2. The method of claim 1 wherein said locus is located on a human Y chromosome.

3. The method of claim 1 wherein said base composition of said previously unknown STR allele is added to said plurality of database stored base compositions.

4. The method of claim 3 wherein said base composition of said previously unknown STR allele comprises a single nucleotide polymorphism relative to a known STR allele.

5-55. (canceled)

56. A purified oligonucleotide primer pair for identifying a known STR allele or characterizing a previously unknown STR allele in a nucleic acid sample, said primer pair configured to produce an amplification product of at least a portion of an STR locus, said amplification product duplicating the sequence of said known STR allele or said previously unknown STR allele, each member of said primer pair having at least 70%, at least 80%, at least 90%, at least 95% or at least 100% sequence identity with a corresponding member of a primer pair selected from the group consisting of: SEQ ID NOs: 16:28, 51:17, 45:60, 10:27, 42:27, 10:35, 24:46, 23:15, 23:5, 24:47, 59:20, 21:49, 59:49, 39:68, 32:50, 19:13, 19:48, 70:57, 26:11, 53:29, 25:18, 69:18, 1:43, 63:54, 67:12, 62:64, 65:44, 36:14, 8:14, 38:61, 36:37, 7:56, 71:41, 22:6, 71:9, 3:58, 2:40, 4:52, 2:52, 62:55, 33:31, 34:30, 73:74, 42:66 and 72:67.

57. The primer pair of claim 56 wherein at least one member of said primer pair comprises a mass-modified nucleobase, universal nucleobase, or a non-templated 5′-thymidine residue or any combination thereof.

58. The primer pair of claim 56 wherein said locus is selected from the group consisting of DYS393, DYS19, DYS391, DYS385a/b, DYS390, DYS392, DYS437, DYS438, DYS439, DYS389I and DYS389II.

59-101. (canceled)

102. A kit comprising one or more purified oligonucleotide primer pairs for identifying a known STR allele or characterizing a previously unknown STR allele in a nucleic acid sample, said one or more primer pairs configured to produce an amplification product of an STR locus, said amplification product duplicating the sequence of said known STR allele or said previously unknown STR allele, each member of said one or more primer pairs having at least 70%, at least 80%, at least 90%, at least 95% or at least 100% sequence identity with a corresponding member of one or more primer pairs selected from the group consisting of: SEQ ID NOs: 16:28, 51:17, 45:60, 10:27, 42:27,110:35, 24:46, 23:15, 23:5, 24:47, 59:20, 21:49, 59:49, 39:68, 32:50, 19:13, 19:48, 70:57, 26:11, 53:29, 25:18, 69:18, 1:43, 63:54, 67:12, 62:64, 65:44, 36:14, 8:14, 38:61, 36:37, 7:56, 71:41, 22:6, 71:9, 3:58, 2:40, 4:52, 2:52, 62:55, 33:31, 34:30, 73:74, 42:66 and 72:67.

103. The kit of claim 102 wherein said one or more primer pairs are contained within the same reaction vessel.

104. The kit of claim 103 wherein said reaction vessel is a well of a 96-well plate.

105. The kit of claim 104 wherein said well comprises five primer pairs, each member of said live primer pairs having at least 70%, at least 80%, at least 90%, at least 95% Or at least 100% sequence identity with a corresponding member of SEQ ID NOs: 23:5, 53:29, 19:48, 63:54 and 39:68.

106. The kit of claim 105 further comprising at least a first additional well comprising four primer pairs each member of said five primer pairs having at least 70%, at least 80%, at least 90%, at least 95% or at least 100% sequence identity with a corresponding member of SEQ ID NOs: 24:47, 22:6, 4:52 and 36:37.

107. The kit of claim 106 further comprising at least a second additional well comprising a primer pair, each member of said primer pair having at least 70%, at least 80%, at least 90%, at least 95% or at least 100% sequence identity with a corresponding member of SEQ ID NOs: 51:17.

108. The kit of claim 107 further comprising at least a third additional well comprising a primer pair, each member of said primer pair having at least 70%, at least 80%, at least 90%, at least 95% or at least 100% sequence identity with a. corresponding member of SEQ ID NOs: 72:67.

109. (canceled)

110. A method of identifying an individual comprising:
a) obtaining a sample from said individual, said sample comprising DNA originating from said individual:
b) identifying a plurality of STR alleles of said DNA according to the method of claim 1, said plurality of STR alleles providing an allelic profile for said individual; and
c) comparing said allelic profile of said individual with a plurality of database-stored allelic profiles of known individuals wherein a match between said allelic profile and a member of said plurality of database-stored allelic profiles identifies said individual.

111. The method of claim 110 wherein a plurality of amplification products are produced in the same reaction vessel.

112. The method of claim 111 wherein said reaction vessel is a well of a 96-well plate.

113. The method of claim 112 wherein said plurality of amplification products comprises five amplification products produced with five primer pairs, each member of said five primer pairs having at least 70%, at least 80%, at least 90%, at least 95% or at least 100% sequence identity with a corresponding member of SEQ ID NOs: 23:5, 53:29, 19:48, 63:54 and 39:68.

114. The method of claim 113 further comprising producing four additional amplification products in at least one additional reaction vessel, said four additional amplification products produced with four primer pairs, each member of said four primer pairs having at least 70%, at least 80%, at least 90%, at least 95% or at least 100% sequence identity with a corresponding member of SEQ ID NOs: 24:47, 22:6, 4:52, and 36:37.

115. The method of claim 114 further comprising producing two additional amplification products in separate reaction vessels with two primer pairs, each member of said two primer pairs having at least 70%, at least 80%, at least 90%, at least 95% or at least 100% sequence identity with a corresponding member of SEQ ID NOs: 51:17 and 72:67.

116-118. (canceled)


SEQUENCE LISTING (74 sequences)

SEQ ID NO: 1 - DNA, 28 bp, Artificial Sequence
  Oligonucleotide Primer
taatgtggtcttctacttgtgtcaatac

SEQ ID NO: 2 - DNA, 32 bp, Artificial Sequence
  Oligonucleotide Primer
tacataggtggagacagatagatgataaatag

SEQ ID NO: 3 - DNA, 30 bp, Artificial Sequence
  Oligonucleotide Primer
tagatacataggtggagacagatagatgat

SEQ ID NO: 4 - DNA, 32 bp, Artificial Sequence
  Oligonucleotide Primer
tagatagatacataggtggagacagatagatg

SEQ ID NO: 5 - DNA, 31 bp, Artificial Sequence
  Oligonucleotide Primer
tagatagattgatagagggagggatagatag

SEQ ID NO: 6 - DNA, 20 bp, Artificial Sequence
  Oligonucleotide Primer
tagcctgggcaacaagagtg

SEQ ID NO: 7 - DNA, 24 bp, Artificial Sequence
  Oligonucleotide Primer
tagtggggaatagttgaacggtaa

SEQ ID NO: 8 - DNA, 19 bp, Artificial Sequence
  Oligonucleotide Primer
tatgggcgtgagtgcatgc

SEQ ID NO: 9 - DNA, 23 bp, Artificial Sequence
  Oligonucleotide Primer
tatttcagcctgggcaacaagag

SEQ ID NO: 10 - DNA, 32 bp, Artificial Sequence
  Oligonucleotide Primer
tcaacaaagaaaagaaatgaaattcagaaagg

SEQ ID NO: 11 - DNA, 23 bp, Artificial Sequence
  Oligonucleotide Primer
tcaacctaccaatcccattcctt

SEQ ID NO: 12 - DNA, 31 bp, Artificial Sequence
  Oligonucleotide Primer
tcaagtccaaaaaatgaggtatgtctcatag

SEQ ID NO: 13 - DNA, 26 bp, Artificial Sequence
  Oligonucleotide Primer
tcaattgccatagagggataggtagg

SEQ ID NO: 14 - DNA, 34 bp, Artificial Sequence
  Oligonucleotide Primer
tcacagatgatatagatagatagataaccacaga

SEQ ID NO: 15 - DNA, 30 bp, Artificial Sequence
  Oligonucleotide Primer
tcacagttatccctgagtagtagaagaatg

SEQ ID NO: 16 - DNA, 31 bp, Artificial Sequence
  Oligonucleotide Primer
tcactatgactactgagtttctgttatagtg

SEQ ID NO: 17 - DNA, 25 bp, Artificial Sequence
  Oligonucleotide Primer
tcatctgggttaaggagagtgtcac

SEQ ID NO: 18 - DNA, 29 bp, Artificial Sequence
  Oligonucleotide Primer
tcattaaacctaccaatcccattccttag

SEQ ID NO: 19 - DNA, 29 bp, Artificial Sequence
  Oligonucleotide Primer
tcattcaatcatacacccatatctgtctg

SEQ ID NO: 20 - DNA, 29 bp, Artificial Sequence
  Oligonucleotide Primer
tcattgcaatgtgtatactcagaaacaag

SEQ ID NO: 21 - DNA, 23 bp, Artificial Sequence
  Oligonucleotide Primer
tcatttttgggccctgcattttg

SEQ ID NO: 22 - DNA, 28 bp, Artificial Sequence
  Oligonucleotide Primer
tccaaaattagtggggaatagttgaacg

SEQ ID NO: 23 - DNA, 32 bp, Artificial Sequence
  Oligonucleotide Primer
tccaactctcatctgtattatctatgtatctg

SEQ ID NO: 24 - DNA, 30 bp, Artificial Sequence
  Oligonucleotide Primer
tccaactctcatctgtattatctatgtgtg

SEQ ID NO: 25 - DNA, 29 bp, Artificial Sequence
  Oligonucleotide Primer
tccaagaaggaaaacaaattttttccttg

SEQ ID NO: 26 - DNA, 23 bp, Artificial Sequence
  Oligonucleotide Primer
tccaagccaagaaggaaaacaaa

SEQ ID NO: 27 - DNA, 31 bp, Artificial Sequence
  Oligonucleotide Primer
tccaattacatagtcctcctttctttttctc

SEQ ID NO: 28 - DNA, 24 bp, Artificial Sequence
  Oligonucleotide Primer
tccatctgggttaaggagagtgtc

SEQ ID NO: 29 - DNA, 26 bp, Artificial Sequence
  Oligonucleotide Primer
tccattaaacctaccaatcccattcc

SEQ ID NO: 30 - DNA, 25 bp, Artificial Sequence
  Oligonucleotide Primer
tcccactcaagtccaaaaaatgagg

SEQ ID NO: 31 - DNA, 28 bp, Artificial Sequence
  Oligonucleotide Primer
tccccaaaaaatgaggtatgtctcatag

SEQ ID NO: 32 - DNA, 27 bp, Artificial Sequence
  Oligonucleotide Primer
tcccttcattcaatcatacacccatat

SEQ ID NO: 33 - DNA, 29 bp, Artificial Sequence
  Oligonucleotide Primer
tcctcttctacttgtgtcaatacagatag

SEQ ID NO: 34 - DNA, 26 bp, Artificial Sequence
  Oligonucleotide Primer
tcctggtcttctacttgtgtcaatac

SEQ ID NO: 35 - DNA, 30 bp, Artificial Sequence
  Oligonucleotide Primer
tcctttctttctctttcctctttctctttc

SEQ ID NO: 36 - DNA, 19 bp, Artificial Sequence
  Oligonucleotide Primer
tcgtgagtgcatgcccatc

SEQ ID NO: 37 - DNA, 33 bp, Artificial Sequence
  Oligonucleotide Primer
tcgttcatagataagtagatagacatcattcac

SEQ ID NO: 38 - DNA, 20 bp, Artificial Sequence
  Oligonucleotide Primer
tctatgggcgtgagtgcatg

SEQ ID NO: 39 - DNA, 29 bp, Artificial Sequence
  Oligonucleotide Primer
tctgcattttggtaccccataatatattc

SEQ ID NO: 40 - DNA, 26 bp, Artificial Sequence
  Oligonucleotide Primer
tctggcttggaattcttttacccatc

SEQ ID NO: 41 - DNA, 23 bp, Artificial Sequence
  Oligonucleotide Primer
tctgggcaacaagagtgaaactc

SEQ ID NO: 42 - DNA, 30 bp, Artificial Sequence
  Oligonucleotide Primer
tgaaagagaaagaggaaagagaaagaaagg

SEQ ID NO: 43 - DNA, 30 bp, Artificial Sequence
  Oligonucleotide Primer
tgaactcaagtccaaaaaatgaggtatgtc

SEQ ID NO: 44 - DNA, 31 bp, Artificial Sequence
  Oligonucleotide Primer
tgaccctgtcattcacagatgatatagatag

SEQ ID NO: 45 - DNA, 31 bp, Artificial Sequence
  Oligonucleotide Primer
tgactactgagtttctgttatagtgtttttt

SEQ ID NO: 46 - DNA, 30 bp, Artificial Sequence
  Oligonucleotide Primer
tgatagattgatagagggagggatagatag

SEQ ID NO: 47 - DNA, 33 bp, Artificial Sequence
  Oligonucleotide Primer
tgatgagagttggatacagaagtaggtataatg

SEQ ID NO: 48 - DNA, 21 bp, Artificial Sequence
  Oligonucleotide Primer
tgcaagcaattgccatagagg

SEQ ID NO: 49 - DNA, 30 bp, Artificial Sequence
  Oligonucleotide Primer
tgcaatgtgtatactcagaaacaaggaaag

SEQ ID NO: 50 - DNA, 23 bp, Artificial Sequence
  Oligonucleotide Primer
tgcatagccaaatatctcctggg

SEQ ID NO: 51 - DNA, 31 bp, Artificial Sequence
  Oligonucleotide Primer
tgcctactgagtttctgttatagtgtttttt

SEQ ID NO: 52 - DNA, 28 bp, Artificial Sequence
  Oligonucleotide Primer
tgcttggaattcttttacccatcatctc

SEQ ID NO: 53 - DNA, 30 bp, Artificial Sequence
  Oligonucleotide Primer
tggaaaacaaattttttccttgtatcacca

SEQ ID NO: 54 - DNA, 25 bp, Artificial Sequence
  Oligonucleotide Primer
tggaactcaagtccaaaaaatgagg

SEQ ID NO: 55 - DNA, 29 bp, Artificial Sequence
  Oligonucleotide Primer
tggaggtatgtctcatagaaaagacatac

SEQ ID NO: 56 - DNA, 21 bp, Artificial Sequence
  Oligonucleotide Primer
tggaggttgtggtgagtcgag

SEQ ID NO: 57 - DNA, 23 bp, Artificial Sequence
  Oligonucleotide Primer
tggataggtaggcaggcagatag

SEQ ID NO: 58 - DNA, 22 bp, Artificial Sequence
  Oligonucleotide Primer
tggcctggcttggaattctttt

SEQ ID NO: 59 - DNA, 20 bp, Artificial Sequence
  Oligonucleotide Primer
tgggccctgcattttggtac

SEQ ID NO: 60 - DNA, 26 bp, Artificial Sequence
  Oligonucleotide Primer
tgggttaaggagagtgtcactatatc

SEQ ID NO: 61 - DNA, 36 bp, Artificial Sequence
  Oligonucleotide Primer
tggtaaatatcattcatagataagtagatagacatc

SEQ ID NO: 62 - DNA, 29 bp, Artificial Sequence
  Oligonucleotide Primer
tggtcttctacttgtgtcaatacagatag

SEQ ID NO: 63 - DNA, 26 bp, Artificial Sequence
  Oligonucleotide Primer
tggtggtcttctacttgtgtcaatac

SEQ ID NO: 64 - DNA, 28 bp, Artificial Sequence
  Oligonucleotide Primer
tgtccaaaaaatgaggtatgtctcatag

SEQ ID NO: 65 - DNA, 19 bp, Artificial Sequence
  Oligonucleotide Primer
tgtgagtgcatgcccatcc

SEQ ID NO: 66 - DNA, 34 bp, Artificial Sequence
  Oligonucleotide Primer
tgtgggataatctatctattccaattacatagtc

SEQ ID NO: 67 - DNA, 31 bp, Artificial Sequence
  Oligonucleotide Primer
tgtggtcttctacttgtgtcaatacagatag

SEQ ID NO: 68 - DNA, 32 bp, Artificial Sequence
  Oligonucleotide Primer
tgtgtatactcagaaacaaggaaagatagata

SEQ ID NO: 69 - DNA, 30 bp, Artificial Sequence
  Oligonucleotide Primer
tgttatttaaaagccaagaaggaaaacaaa

SEQ ID NO: 70 - DNA, 28 bp, Artificial Sequence
  Oligonucleotide Primer
ttcaatcatacacccatatctgtctgtc

SEQ ID NO: 71 - DNA, 26 bp, Artificial Sequence
  Oligonucleotide Primer
ttggggaatagttgaacggtaaacag

SEQ ID NO: 72 - DNA, 31 bp, Artificial Sequence
  Oligonucleotide Primer
tttaaagagaaagaggaaagagaaagaaagg

SEQ ID NO: 73 - DNA, 33 bp, Artificial Sequence
  Oligonucleotide Primer
tttccattttggtaccccataatatattctatc

SEQ ID NO: 74 - DNA, 34 bp, Artificial Sequence
  Oligonucleotide Primer
ttttgtatactcagaaacaaggaaagatagatag